(19) United States
(12) Patent Application Publication
(10) Pub. No.: US 2022/0115143 A1
(43) Pub. Date: Apr. 14, 2022

(54) Systems and Methods for determining the Contribution of a Given Measurement to a Patient State Determination

(71) Applicant: Etiometry, Inc., Boston, MA (US)

(72) Inventors: Dimitar V. Baronov, Weston, MA (US); Michael F. McManus, Pembroke, MA (US)

(21) Appl. No.: 17/501,978

(22) Filed: Oct. 14, 2021

Related U.S. Application Data
(60) Provisional application No. 63/091,427, filed on Oct. 14, 2020.

(51) Int. Cl. G16H 50/30; G16H 10/60; G16H 40/67

(52) U.S. Cl. CPC G16H 50/30; G06N 7/005; G16H 40/67; G16H 10/60

(57) ABSTRACT

Systems and methods produce a quantitative indication of the influence, on determination of a patient's clinical risk, of one or more measurements of one or more internal state variables. Illustrative embodiments compute a reference patient's clinical risk of being in a specific patient state using measurements of measurable internal state variables, and compute alternates of the same clinical risk using alternate values for at least one of the measurements of measurable internal state variables, and determine which of the measurements of internal state variables have the greatest quantitative impact on the patient's clinical risk by comparing the reference clinical risk to the alternate clinical risks.

### RELATED APPLICATIONS

This application claims priority to U.S. provisional patent application Ser. No. 63/091,427, filed Oct. 14, 2020 and titled “Systems and Methods for determining the Contribution of a Given Measurement to a Patient State Determination (Measured Contribution),” naming Dimitar V. Baronov and Michael F. McManus as inventors [Attorney Docket No. 3816-12101], the entire subject matter of which is incorporated herein by this reference for all purposes.

This application is also related to each of the following patent applications:

U.S. Continuation-in-Part application Ser. No. 17/033,591 filed Sep. 25, 2020 and entitled “Systems and Methods for Transitioning Patient Care from Signal-Based Monitoring to Risk-Based Monitoring” [Attorney Docket No. 3816-10610], U.S. provisional application Ser. No. 62/906,518 filed Sep. 26, 2019 and entitled “Systems and Methods for Transitioning Patient Care from Signal-Based Monitoring to Risk-Based Monitoring” [Attorney Docket No. 3816-10608],

U.S. continuation application Ser. No. 17/064,248 filed Oct. 6, 2020 and entitled “Systems and Methods for Transitioning Patient Care from Signal-Based Monitoring to Risk-Based Monitoring [Attorney Docket No. 3816-11903],

U.S. non-provisional patent application Ser. No. 16/113,486 filed Aug. 27, 2018 and entitled “Systems and Methods for Transitioning Patient Care from Signal-Based Monitoring to Risk-Based Monitoring,” [Attorney Docket No. 3816-11901],

U.S. non-provisional patent application Ser. No. 14/727,696, filed Jun. 1, 2015 and entitled “Systems and Methods for Transitioning Patient Care from Signal-Based Monitoring to Risk-Based Monitoring,” issued Aug. 28, 2018 as U.S. Pat. No. 10,062,456 [Attorney Docket No. 3816-11701],

U.S. non-provisional patent application Ser. No. 13/826,441, filed Mar. 14, 2013 and entitled “Systems and Methods for Transitioning Patient Care from Signal-Based Monitoring to Risk-Based Monitoring, Attorney Docket No. 3816-11601],

U.S. patent application Ser. No. 13/689,029, filed on Nov. 29, 2012, entitled SYSTEMS AND METHODS FOR OPTIMIZING MEDICAL CARE THROUGH DATA MONITORING AND FEEDBACK TREATMENT [Attorney Docket No. 3816-10501]; and

U.S. application Ser. No. 13/328,411, filed on Dec. 16, 2011, entitled METHOD AND APPARATUS FOR VISUALIZING THE RESPONSE OF A COMPLEX SYSTEM TO CHANGES IN A PLURALITY OF INPUTS [Attorney Docket No. 3816-10701];

and also to the following provisional patent applications:

U.S. Provisional Application No. 61/727,820, filed on Nov. 19, 2012, entitled USER INTERFACE DESIGN FOR RAHM [Attorney Docket No. 3816-10401;

U.S. Provisional Application No. 61/699,492, filed on Sep. 11, 2012, entitled SYSTEMS AND METHODS FOR EVALUATING CLINICAL TRAJECTORIES AND TREATMENT STRATEGIES FOR OUTPATIENT CARE [Attorney Docket No. 3816-10301];

U.S. Provisional Application No. 61/684,241, filed on Aug. 17, 2012, entitled SYSTEM AND METHODS FOR PROVIDING RISK ASSESSMENT IN ASSISTING CLINICIANS WITH EFFICIENT AND EFFECTIVE BLOOD MANAGEMENT [Attorney Docket No. 3816-10101];

U.S. Provisional Application No. 61/620,144, filed on Apr. 4, 2012, entitled SYSTEMS AND METHODS FOR PROVIDING MOBILE ADVANCED CARDIAC SUPPORT [Attorney Docket No. 3816-11201];

U.S. Provisional Application No. 61/614,861, filed on Mar. 23, 2012 entitled SYSTEMS AND METHODS FOR REDUCING MORBIDITY AND MORTALITY WHILE REDUCING LENGTH OF STAY IN A HOSPITAL SETTING [Attorney Docket No. 3816-11101];

U.S. Provisional Application No. 61/614,846, filed Mar. 23, 2012, entitled SYSTEMS AND METHODS FOR PROVIDING MOBILE ADVANCED CARDIAC SUPPORT [Attorney Docket No. 3816-11001];

and

U.S. Provisional Application No. 61/774,274, filed on Mar. 7, 2013, entitled SYSTEMS AND METHODS FOR TRANSITIONING PATIENT CARE FROM SIGNAL-BASED MONITORING TO RISK-BASED MONITORING [Attorney Docket No. 3816-10201],

the entire subject matter of each of the foregoing applications being incorporated herein by this reference for all purposes.

### STATEMENT REGARDING FEDERALLY SPONSORED RESEARCH OR DEVELOPMENT

This invention was made with government support under R44HL117340 awarded by the National Heart, Lung, And Blood Institute of the National Institutes of Health. The government has certain rights in the invention.

### BACKGROUND ART

The present disclosure relates to systems and methods for risk-based patient monitoring. More particularly, the present disclosure relates to systems and methods for assessing the current and future risks of a patient by combining data of the patient from various different sources.

Practicing medicine is becoming increasingly more complicated due to the introduction of new sensors and treatments. As a result, clinicians are confronted with an avalanche of patient data, which needs to be evaluated and well understood in order to prescribe the optimal treatment from the multitude of available options, while reducing patient risks. One environment where this avalanche of information has become increasingly problematic is the Intensive Care Unit (ICU). There, the experience of the attending physician and the physician's ability to assimilate the available physiologic information have a strong impact on the clinical outcome. It has been determined that hospitals which do not maintain trained intensivists around the clock experience a 14.4% mortality rate as opposed to a 6.0% rate for fully staffed centers. It is estimated that raising the level of care to that of average trained physicians across all ICUs can save 160,000 lives and 54.3 Bn annually. As of 2012, there is a shortage of intensivists, and projections estimate the shortage will only worsen, reaching a level of 35% by 2020.

The value of experience in critical care can be explained by the fact that clinical data in the ICU is delivered at a rate far greater than even the most talented physician can absorb, and studies have shown that errors are six times more likely under conditions of information overload and eleven time more likely with an acute time shortage. Moreover, treatment decisions in the ICU heavily rely on clinical signs that are not directly measurable, but are inferred from other physiologic information. Thus clinician expertise and background play a more significant role in the minute to minute decision making process. Not surprisingly, this leads to a large variance in hidden parameter estimation. As an example, although numerous proxies for cardiac output are continuously monitored in critical care, studies have demonstrated poor correlation between subjective assessment by clinicians, and objective measurement by thermodilution. Experienced intensivists incorporate this inherent uncertainty in their decision process by effectively conducting risk management, i.e. prescribing the treatment not only based on the most probable patient state, but also weighing in the risks of the patient being in other more adverse states. From this perspective, experienced intensivists confront the data overload in intensive care by converting the numerous heterogeneous signals from patient observations into a risk assessment.

Therefore, there is a clear need for a decision support system in the ICU that achieves a paradigm shift from signal-based patient monitoring to risk-based patient monitoring, and consequently helps physicians overcome the barrage of data in the ICU.

### BRIEF SUMMARY

An illustrative embodiment provides a method that determines which measurement, of a plurality of measurements of measurable internal state variables of a patient, has the greatest quantitative impact on determination of the patient's patient state. For example, an illustrative embodiment computes a quantitative reference risk that the patient is in a specified patient state based on an initial set of measurements of measurable internal state variables of the patient.

The embodiment also computes a first alternate quantitative risk that the patient is in the specified patient state by substituting a first alternate measurement for one of the measurements of measurable internal state variables of the patient, thereby creating a first alternate set of measurements, and computing the first alternate quantitative risk using the first alternate second set of measurements.

The embodiment also computes a second alternate quantitative risk that the patient is in the specified patient state by substituting a second alternate measurement for another one of the measurements of measurable internal state variables of the patient, thereby creating a second alternate second set of measurements, and computing the second alternate quantitative risk using the second alternate second set of measurements.

The embodiment then determines which measurement, of the initial set of measurements, has the largest quantitative impact on the quantitative reference risk. Specifically, the embodiment compares the reference risk to the first alternate risk (which is associated with the first alternate measurement) and then to the second alternate risk (which is associated with the second alternate measurement) to determine of the first alternate risk and the second alternate risk has the largest difference (or “delta”) from the reference risk. The measurement of the internal state variable associated with the alternate risk that has the largest difference (or “delta”) from the reference risk is the measurement that has largest quantitative impact on the quantitative reference risk.

For example, in one embodiment a method of transforming measured data of a patient into data for a particular patient state based on a generated internal state variable, includes:

providing a plurality of sensors including at least a first sensor and a second sensor, to measure a corresponding plurality of internal state variables, the plurality of sensors physically attached to the patient;

substantially continuously acquiring, by a computer over a series of time steps t_{K}, K=0, 1, . . . Z, from the plurality of sensors connected with the patient, a set of as-measured datums m_{S}, S=1, 2 of internal state variables, including a first as-measured datum (m₁) for a first internal state variable (V₁) at time step tₖ₊₁, and a second as-measured datum (m₂) for a second internal state variable (V₂) at time step tₖ₊₁;

generating, by the computer using the set of as-measured datums from time step tₖ₊₁, a reference conditional likelihood kernel for the internal state variables at time tₖ₊₁, the reference conditional likelihood kernel including a set of probability density functions of the internal state variables for the time step tₖ₊₁, each of the internal state variables describing a parameter physiologically relevant to the particular patient state of said patient at time step tₖ₊₁;

generating, with the computer and using Bayes theorem, reference posterior predicted conditional probability density functions for the plurality of the internal state variables for the time step tₖ given the reference conditional likelihood kernel for the internal state variables at time tₖ₊₁ and predicted probability density functions of each of the internal state variables predicted from a preceding time step tₖ for time step tₖ₊₁; and

generating, from the reference posterior predicted conditional probability density functions, a reference function of the generated internal state variable;

identifying, with the computer, from the reference function of the generated internal state variable, a reference risk that the patient is in the particular patient state;

and by

editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A});

generating, by the computer using the first alternate set of datums, a first alternate conditional likelihood kernel for the internal state variables at time tₖ₊₁, the first alternate conditional likelihood kernel including a first alternate set of probability density functions of the internal state variables for the time step tₖ₊₁;

generating, with the computer and using Bayes theorem, first alternate posterior predicted conditional probability density functions for the plurality of the internal state variables for the time step tₖ₊₁ given the first alternate conditional likelihood kernel for the internal state variables at time tₖ₊₁ and the predicted probability density functions of each of the internal state variables for time step tₖ₊₁;

generating, from the first alternate posterior predicted conditional probability density functions, a first alternate function of the generated internal state variable; and

identifying, with the computer, from the first alternate function of the generated internal state variable, a first alternate risk that the patient is in the particular patient state at time step tₖ₊₁, said first alternate risk associated with said first internal state variable V₁;

and

editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A});

generating, by the computer using the second alternate set of datums, a second alternate conditional likelihood kernel for the internal state variables at time tₖ₊₁, the second alternate conditional likelihood kernel including a second alternate set of probability density functions of the internal state variables for the time step tₖ₊₁;

generating, with the computer and using Bayes theorem, second alternate posterior predicted conditional probability density functions for the plurality of the internal state variables for the time step tₖ₊₁ given the second alternate conditional likelihood kernel for the internal state variables at time tₖ₊₁ and the predicted probability density functions of each of the internal state variables for time step tₖ₊₁; and

generating, from the second posterior predicted conditional probability density functions, a second alternate function of the generated internal state variable; and

identifying, with the computer, from the second alternate function of the generated internal state variable, a second alternate risk that the patient is in the particular patient state at time step tₖ₊₁, said second alternate risk associated with said second internal state variable (V₂);

determining, as among the as-measured datums, which as-measured datum has the quantitatively greatest influence on the reference risk (that the patient is in the particular patient state at time step tₖ₊₁) by:

comparing the first alternate risk that the patient is in the particular patient state to the reference risk to produce a first delta associated with the first internal state variable, and by

comparing the second alternate risk that the patient is in the particular patient state to the reference risk to produce a second delta associated with the second internal state variable,

the as-measured datum having the quantitatively greatest influence on the reference risk being the as-measured datum associated with the larger of the first delta and the second delta; and

displaying, on a graphical user interface, the reference risk that the patient is in the particular patient state at time step tₖ₊₁, and an identifier of the as-measured datum has the quantitatively greatest influence on the reference risk at time step tₖ₊₁.

In some embodiments:

the particular patient state is hyperlactatemia;

the first sensor is a heart rate sensor;

the second sensor is an SpO₂ sensor;

the internal state variable is a hidden internal state variable: whole blood lactate level;

the first internal state variable (V₁) is the patient's heart rate at time step tₖ₊₁;

the second internal state variable (V₂) is the patient's SpO2 at time step tₖ₊₁; and wherein:

identifying, with the computer, from the function of the internal state variable, a reference risk that the patient is in the particular patient state of hyperlactatemia includes determining the cumulative distribution of whole blood lactate level above a predetermined threshold.

In some such embodiments, the first alternate datum value to produce a first alternate datum (m_{1A}) includes a datum value selected from one of a nominal heart and a null value for the heart rate.

In other such embodiments, the second alternate datum value to produce a second alternate datum (m_{2A}) includes a datum value selected from one of: a nominal value of SpO2 and a null value of SpO₂.

In another embodiments, the particular patient state is inadequate ventilation of carbon dioxide;

the first sensor is a heart rate sensor;

the second sensor is an SpO₂ sensor;

the internal state variable is a hidden internal state variable: arterial partial pressure of carbon dioxide blood [p(PaCO2)];

the first internal state variable (V₁) is the patient's heart rate at time step tₖ₊₁;

the second internal state variable (V₂) is the patient's SpO2 at time step tₖ₊₁; and wherein:

identifying, with the computer, from the function of the internal state variable, a reference risk that the patient is in the particular patient state of inadequate ventilation of carbon dioxide includes determining the cumulative distribution of p(PaCO2)] above a predetermined threshold.

In some such embodiments, the first alternate datum value to produce a first alternate datum (m1A) includes a datum value selected from one of a nominal heart and a null value for the heart rate.

In other such embodiments, the second alternate datum value to produce a second alternate datum (m2A) includes a datum value selected from one of: a nominal value of SpO2 and a null value of SpO2.

In another embodiment, the set of sensors further includes a third sensor, and

the particular patient state is acidosis;

the first sensor is a heart rate sensor;

the second sensor is an SpO₂ sensor;

the third sensor is a respiratory rate sensor;

the internal state variable is a hidden internal state variable: arterial blood pH;

the first internal state variable (V₁) is the patient's heart rate at time step tₖ₊₁;

the second internal state variable (V₂) is the patient's SpO2 at time step tₖ₊₁;

the third internal state variable (V₃) is the patient's respiratory rate; and wherein:

identifying, with the computer, from the function of the internal state variable, a reference risk that the patient is in the particular patient state of acidosis includes determining the cumulative distribution of Arterial blood pH below a predetermined threshold.

In some such embodiments, the first alternate datum value to produce a first alternate datum (m1A) includes a datum value selected from one of a nominal heart and a null value for the heart rate.

In other embodiments, the second alternate datum value to produce a second alternate datum (m2A) includes a datum value selected from one of: a nominal value of SpO2 and a null value of SpO2.

In other embodiments, substantially continuously acquiring, by a computer over a series of time steps t_{K}, K=0, 1, . . . Z, from the plurality of sensors connected with the patient, a set of as-measured datums m_{S}, S=1, 2 of internal state variables further includes acquiring a third as-measured datum (m₃) for a third internal state variable (V₃) at time step tₖ₊₁; and the method further includes:

editing the set of as-measured datums by replacing the third as-measured datum (m₃) with a third alternate datum value to produce a third alternate datum (m_{3A}), the third alternate datum value distinct from the as-measured value of the third as-measured datum (m₃), to produce a third alternate set of datums including the first as-measured datum (m₁) and the second as-measured datum (m₂) and the third alternate datum (m_{3A});

generating, by the computer using the third alternate set of datums, a third alternate conditional likelihood kernel for the internal state variables at time tₖ₊₁, the third alternate conditional likelihood kernel including a third alternate set of probability density functions of the internal state variables for the time step tₖ₊₁;

generating, with the computer and using Bayes theorem, third alternate posterior predicted conditional probability density functions for the plurality of the internal state variables for the time step tₖ₊₁ given the third alternate conditional likelihood kernel for the internal state variables at time tₖ₊₁ and the predicted probability density functions of each of the internal state variables for time step tₖ₊₁;

generating, from the third alternate posterior predicted conditional probability density functions, a third alternate function of the generated internal state variable; and

identifying, with the computer, from the third alternate function of the generated internal state variable, a third alternate risk that the patient is in the particular patient state at time step tₖ₊₁, said third alternate risk associated with said third internal state variable V₃; and wherein:

determining, as among the as-measured datums, which as-measured datum has the quantitatively greatest influence on the reference risk (that the patient is in the particular patient state at time step tₖ₊₁) includes:

comparing the first alternate risk that the patient is in the particular patient state to the reference risk to produce a first delta associated with the first internal state variable, and by

comparing the second alternate risk that the patient is in the particular patient state to the reference risk to produce a second delta associated with the second internal state variable,

comparing the third alternate risk that the patient is in the particular patient state to the reference risk to produce a third delta associated with the first internal state variable, and wherein

the as-measured datum having the quantitatively greatest influence on the reference risk being the as-measured datum associated with the larger of the first delta and the second delta and the third delta.

In yet another embodiments, the set of sensors further includes a third sensor, and:

the particular patient state is inadequate oxygen delivery;

the first sensor is a heart rate sensor;

the second sensor is an SpO₂ sensor;

the third sensor is a respiratory rate sensor;

the internal state variable is a hidden internal state variable: mixed venous oxygen saturation;

the first internal state variable (V₁) is the patient's heart rate at time step tₖ₊₁;

the second internal state variable (V₂) is the patient's SpO2 at time step tₖ₊₁;

the third internal state variable (V₃) is the patient's respiratory rate; and wherein:

identifying, with the computer, from the function of the internal state variable, a reference risk that the patient is in the particular patient state of inadequate oxygen delivery includes determining the cumulative distribution of cumulative distribution mixed venous oxygen saturation below a predetermined threshold.

In some such embodiments, the first alternate datum value to produce a first alternate datum (m1A) includes a datum value selected from one of a nominal heart and a null value for the heart rate.

In other such embodiments, the second alternate datum value to produce a second alternate datum (m2A) includes a datum value selected from one of: a nominal value of SpO2 and a null value of SpO2.

In other embodiments, substantially continuously acquiring, by a computer over a series of time steps t_{K}, K=0, 1, . . . Z, from the plurality of sensors connected with the patient, a set of as-measured datums m_{S}, S=1, 2 of internal state variables further includes acquiring a third as-measured datum (m₃) for a third internal state variable (V₃) at time step tₖ₊₁; and the method further includes:

editing the set of as-measured datums by replacing the third as-measured datum (m₃) with a third alternate datum value to produce a third alternate datum (m_{3A}), the third alternate datum value distinct from the as-measured value of the third as-measured datum (m₃), to produce a third alternate set of datums including the first as-measured datum (m₁) and the second as-measured datum (m₂) and the third alternate datum (m_{3A});

generating, by the computer using the third alternate set of datums, a third alternate conditional likelihood kernel for the internal state variables at time tₖ₊₁, the third alternate conditional likelihood kernel including a third alternate set of probability density functions of the internal state variables for the time step tₖ₊₁;

generating, with the computer and using Bayes theorem, third alternate posterior predicted conditional probability density functions for the plurality of the internal state variables for the time step tₖ₊₁ given the third alternate conditional likelihood kernel for the internal state variables at time tₖ₊₁ and the predicted probability density functions of each of the internal state variables for time step tₖ₊₁;

generating, from the third alternate posterior predicted conditional probability density functions, a third alternate function of the generated internal state variable; and

identifying, with the computer, from the third alternate function of the generated internal state variable, a third alternate risk that the patient is in the particular patient state at time step tₖ₊₁, said third alternate risk associated with said third internal state variable V₃; and wherein:

determining, as among the as-measured datums, which as-measured datum has the quantitatively greatest influence on the reference risk (that the patient is in the particular patient state at time step tₖ₊₁) includes:

comparing the first alternate risk that the patient is in the particular patient state to the reference risk to produce a first delta associated with the first internal state variable, and by

comparing the second alternate risk that the patient is in the particular patient state to the reference risk to produce a second delta associated with the second internal state variable,

comparing the third alternate risk that the patient is in the particular patient state to the reference risk to produce a third delta associated with the first internal state variable, and wherein

the as-measured datum having the quantitatively greatest influence on the reference risk being the as-measured datum associated with the larger of the first delta and the second delta and the third delta.

In another embodiment, a system for transforming measured data of a patient into data for a particular patient state based on a generated internal state variable includes:

a computer including a computer processor;

a display in data communication with the computer processor;

a memory in data communication with the computer processor, the memory holding instructions that, when executed by the computer processor, cause the system to perform a method, the method including:

substantially continuously acquiring, by a computer over a series of time steps t_{K}, K=0, 1, . . . Z, from a plurality of sensors connected with the patient, a set of as-measured datums m_{S}, S=1, 2 of internal state variables, including a first as-measured datum (m₁) for a first internal state variable (V₁) at time step tₖ₊₁, and a second as-measured datum (m₂) for a second internal state variable (V₂) at time step tₖ₊₁;

generating, by the computer using the set of as-measured datums (m₁, m₂) from time step tₖ₊₁, a reference conditional likelihood kernel for the internal state variables at time tₖ₊₁, the reference conditional likelihood kernel including a set of probability density functions of the internal state variables for the time step tₖ₊₁, each of the internal state variables describing a parameter physiologically relevant to the particular patient state of said patient at time step tₖ₊₁;

generating, with the computer and using Bayes theorem, reference posterior predicted conditional probability density functions for the plurality of the internal state variables for the time step tₖ given the reference conditional likelihood kernel for the internal state variables at time tₖ₊₁ and predicted probability density functions of each of the internal state variables predicted from a preceding time step tₖ for time step tₖ₊₁; and

generating, from the reference posterior predicted conditional probability density functions, a reference function of the generated internal state variable;

identifying, with the computer, from the reference function of the generated internal state variable, a reference risk that the patient is in the particular patient state;

and by

editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A});

generating, by the computer using the first alternate set of datums, a first alternate conditional likelihood kernel for the internal state variables at time tₖ₊₁, the first alternate conditional likelihood kernel including a first alternate set of probability density functions of the internal state variables for the time step tₖ₊₁;

generating, with the computer and using Bayes theorem, first alternate posterior predicted conditional probability density functions for the plurality of the internal state variables for the time step tₖ₊₁ given the first alternate conditional likelihood kernel for the internal state variables at time tₖ₊₁ and the predicted probability density functions of each of the internal state variables for time step tₖ₊₁;

generating, from the first alternate posterior predicted conditional probability density functions, a first alternate function of the generated internal state variable; and

identifying, with the computer, from the first alternate function of the generated internal state variable, a first alternate risk that the patient is in the particular patient state at time step tₖ₊₁, said first alternate risk associated with said first internal state variable V₁;

and

editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A});

generating, by the computer using the second alternate set of datums, a second alternate conditional likelihood kernel for the internal state variables at time tₖ₊₁, the second alternate conditional likelihood kernel including a second alternate set of probability density functions of the internal state variables for the time step tₖ₊₁;

generating, with the computer and using Bayes theorem, second alternate posterior predicted conditional probability density functions for the plurality of the internal state variables for the time step tₖ₊₁ given the second alternate conditional likelihood kernel for the internal state variables at time tₖ₊₁ and the predicted probability density functions of each of the internal state variables for time step tₖ₊₁; and

generating, from the second posterior predicted conditional probability density functions, a second alternate function of the generated internal state variable; and

identifying, with the computer, from the second alternate function of the generated internal state variable, a second alternate risk that the patient is in the particular patient state at time step tₖ₊₁, said second alternate risk associated with said second internal state variable (V₂);

determining, as among the as-measured datums, which as-measured datum has the quantitatively greatest influence on the reference risk (that the patient is in the particular patient state at time step tₖ₊₁) by:

comparing the first alternate risk that the patient is in the particular patient state to the reference risk to produce a first delta associated with the first internal state variable, and by

comparing the second alternate risk that the patient is in the particular patient state to the reference risk to produce a second delta associated with the second internal state variable,

the as-measured datum having the quantitatively greatest influence on the reference risk being the as-measured datum associated with the larger of the first delta and the second delta; and

displaying, on a graphical user interface, the reference risk that the patient is in the particular patient state at time step tₖ₊₁, and an identifier of the as-measured datum that has the quantitatively greatest influence on the reference risk at time step tₖ₊₁.

In some such system embodiments:

the particular patient state is hyperlactatemia;

the plurality of sensors includes a heart rate sensor and an SpO₂ sensor;

the internal state variable is a hidden internal state variable: whole blood lactate level;

the first internal state variable (V₁) is the patient's heart rate at time step tₖ₊₁;

the second internal state variable (V₂) is the patient's SpO2 at time step tₖ₊₁; and wherein:

identifying, with the computer, from the function of the internal state variable, a reference risk that the patient is in the particular patient state of hyperlactatemia includes determining the cumulative distribution of whole blood lactate level above a threshold of 4 mmol/L.

In some system embodiments, the particular patient state is inadequate ventilation of carbon dioxide;

the first sensor is a heart rate sensor;

the second sensor is an SpO₂ sensor;

the internal state variable is a hidden internal state variable: arterial partial pressure of carbon dioxide blood [p(PaCO2)];

the first internal state variable (V₁) is the patient's heart rate at time step tₖ₊₁;

the second internal state variable (V₂) is the patient's SpO2 at time step tₖ₊₁; and wherein:

identifying, with the computer, from the function of the internal state variable, a reference risk that the patient is in the particular patient state of inadequate ventilation of carbon dioxide includes determining the cumulative distribution of p(PaCO2)] above a threshold of 50 mmHg.

In some system embodiments, the particular patient state is inadequate oxygen delivery;

the first sensor is a heart rate sensor;

the second sensor is an SpO₂ sensor;

the third sensor is a respiratory rate sensor;

the internal state variable is a hidden internal state variable: mixed venous oxygen saturation;

the first internal state variable (V₁) is the patient's heart rate at time step tₖ₊₁;

the second internal state variable (V₂) is the patient's SpO2 at time step tₖ₊₁;

the third internal state variable (V₃) is the patient's respiratory rate; and wherein:

identifying, with the computer, from the function of the internal state variable, a reference risk that the patient is in the particular patient state of inadequate oxygen delivery includes determining the cumulative distribution of cumulative distribution mixed venous oxygen saturation below 40%.

In some system embodiments, the particular patient state is acidosis;

the first sensor is a heart rate sensor;

the second sensor is an SpO₂ sensor;

the third sensor is a respiratory rate sensor;

the internal state variable is a hidden internal state variable: arterial blood pH;

the first internal state variable (V₁) is the patient's heart rate at time step tₖ₊₁;

the second internal state variable (V₂) is the patient's SpO2 at time step tₖ₊₁;

the third internal state variable (V₃) is the patient's respiratory rate; and wherein:

identifying, with the computer, from the function of the internal state variable, a reference risk that the patient is in the particular patient state of acidosis includes determining the cumulative distribution of arterial blood pH below a threshold of 7.25.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

It should be understood at the outset that although illustrative implementations of one or more embodiments of the present disclosure are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

In the drawings:

FIG. 1A and FIG. 1B illustrate conceptually a medical care risk-based monitoring environment in accordance with the disclosure;

FIG. 2A illustrates conceptually a basic schematic of an embodiment of a physiology observer module in accordance with the disclosure;

FIG. 2B schematically illustrates an embodiment of a predict module;

FIG. 2C schematically illustrates an embodiment of an update module;

FIG. 2D, FIG. 2E and FIG. 2F illustrate conceptually exemplary graphs of probability density functions for select ISVs as generated by the physiology observer module in accordance with the disclosure;

FIG. 2G is a flowchart describing an embodiment of a method of operation of a physiology observer module in accordance with the disclosure;

FIG. 3 illustrates conceptually a non-limiting example of a physiology observer process in accordance with the disclosure;

FIG. 4A illustrates conceptually a non-limiting example of the physiology observer process in accordance with the disclosure;

FIG. 4B illustrates a method applying intermittent laboratory data through the physiology observer module to achieve better accuracy in an estimated ISV PDF;

FIG. 5 illustrates conceptually a timeline, wherein back propagation is used to incorporate information in accordance with the disclosure;

FIG. 6 illustrates conceptually an example of a process involving mean arterial blood pressure (ABPm) in accordance with the disclosure;

FIG. 7 illustrates conceptually an example of resampling in accordance with the disclosure;

FIG. 8A schematically illustrates an embodiment of a clinical trajectory interpreter module using joint Probability Density Functions of ISVs and performing state probability estimation to calculate the probabilities of different patient states in accordance with the disclosure;

FIG. 8B is a flow chart that illustrates an embodiment of a method of operation of a clinical trajectory interpreter module.

FIG. 8C and FIG. 8D schematically illustrate an embodiment of a clinical trajectory interpreter module using joint Probability Density Functions of ISVs to determine the relative contribution of at least one internal state variable to a risk determination via a “measurement contribution” process;

FIG. 8E is a flowchart of an embodiment of a method of assessing measurement contribution;

FIG. 8F schematically illustrates an embodiment of a display showing influence of internal state variables on a patient's clinical risk;

FIG. 9 illustrates conceptually a non-limiting example of a definition of a patient state employed by the clinical trajectory interpreter module in accordance with the disclosure;

FIG. 10A illustrates conceptually a non-limiting example of how a clinical trajectory interpreter module may employ the definition of patient states to assign probabilities that the patient may be classified under each of the four possible patient states at a particular point of time;

FIG. 10B schematically illustrates another embodiment of a clinical trajectory interpreter module using joint Probability Density Functions of ISVs and performing state probability estimation to calculate the probabilities of different patient states in accordance with the disclosure

FIG. 10C, FIG. 10D, FIG. 10E, FIG. 10F and FIG. 10G each schematically illustrates, respectively, a method of determining a patient state using probability density functions of internal state variables;

FIG. 11A schematically illustrates an embodiment of an observation model that may be used to relate the derived variables with the available sensor data in accordance with the disclosure;

FIG. 11B and FIG. 11C schematically illustrate the Gaussian representation of the PDF of the ISVs, and the schematic of the EKF inference engine implementation.

### DETAILED DESCRIPTION OF SPECIFIC EMBODIMENTS

Technologies are provided herein for improving risk-based patient monitoring of individual patients to clinical personnel.

Illustrative embodiments improve the ability of systems and methods to identify and disclose patient risk. A patient's body may be thought of as a complex system or machine having internal state variables, some of which are directly measurable (e.g., they can be measured with sensors) and some of which are hidden.

Multi-variate analysis can combine multiple measurements that do not directly measure hidden internal state variables, to provide risk assessment of the patient being in a particular adverse physiologic state. However, such analysis lacks a way to qualitatively and/or quantitatively assess the influence of a given one (or more) of such measurements on the patient's health or risk of the patient being in a specific patient state. In other words, in an analysis of an internal state variable (including without limitation analysis of a hidden internal state variable) based on quantified measurements of other internal state variables (that are directly measurable by sensors), does not allow a clinician to determine or know which quantified measurement has the quantitatively greatest influence on the patient's particular adverse physiologic patient state. Knowledge of which quantified measurement has the quantitatively greatest influence on the patient's particular adverse physiologic state would allow the clinician to determine which treatment, from among a set of available treatments, to apply to address the internal state variable measured by the quantified measurement that has the quantitatively greatest influence on the patient's particular adverse physiologic state.

To address that shortcoming, systems and methods disclosed herein produce a quantitative indication of the influence, on determination of a patient's clinical risk (as described herein), of one or more measurements of one or more internal state variables.

For example, an illustrative embodiment provides a method that determines which measurement, of a plurality of measurements of measurable internal state variables of a patient, has the greatest quantitative impact on determination of the patient's patient state. For example, an illustrative embodiment computes a quantitative reference risk that the patient is in a specified patient state based on an initial set of measurements of measurable internal state variables of the patient.

The embodiment also computes a first alternate quantitative risk that the patient is in the specified patient state by substituting a first alternate measurement for one of the measurements of measurable internal state variables of the patient, thereby creating a first alternate set of measurements, and computing the first alternate quantitative risk using the first alternate second set of measurements.

The embodiment also computes a second alternate quantitative risk that the patient is in the specified patient state by substituting a second alternate measurement for another one of the measurements of measurable internal state variables of the patient, thereby creating a second alternate second set of measurements, and computing the second alternate quantitative risk using the second alternate second set of measurements.

The embodiment then determines which measurement, of the initial set of measurements, has the largest quantitative impact on the quantitative reference risk. Specifically, the embodiment compares the reference risk to the first alternate risk (which is associated with the first alternate measurement) and then to the second alternate risk (which is associated with the second alternate measurement) to determine of the first alternate risk and the second alternate risk has the largest difference (or “delta”) from the reference risk. The measurement of the internal state variable associated with the alternate risk that has the largest difference (or “delta”) from the reference risk is the measurement that has largest quantitative impact on the quantitative reference risk.

The technologies described herein can be embodied as a monitoring system for critical care, which combines data from various bedside monitors, electronic medical records, and other patient specific information to assess the current and the future risks to the patient. The technologies can be also embodied as a decision support system that prompts the user with specific actions according to a standardized medical plan, when patient specific risks pass a predefined threshold. Yet another embodiment of the described technologies is an outpatient monitoring system which combines patient and family evaluation, together with information about medication regiments and physician evaluations to produce a risk profile of the patient, continuously track its clinical trajectory, and provide decision support to clinicians as regarding when to schedule a visit or additional tests.

### Definitions

As used in this description and the accompanying claims, the following terms shall have the meanings indicated, unless the context otherwise requires.

The term “clinical risk” means the probability of a patient being in a particular patient state, for example at a particular time.

The term “clinical trajectory” means the sequence of patient states through which a patient evolves during a patient's clinical course.

The term “hidden,” in reference to an internal state variable, means an ISV that is not directly measured by a sensor coupled to the patient. Some hidden ISVs cannot be directly measured by a sensor coupled to the patient. Some hidden ISVs require laboratory analysis of a sample (e.g., blood) taken from the patient. As described below, some hidden ISVs may be generated from measurements of ISVs that are not hidden, and may be referred-to as “generated internal state variables.”

The term “internal state variable” (or “ISV”) means a parameter of a patient's physiology that is physiologically relevant to one of a treatment and a condition of a patient.

Examples of ISVs include, without limitation, ISVs that are directly observable with noise (as a non-limiting example, heart rate is a directly observable ISV), ISVs that are hidden (as a non-limiting example, alveolar dead space, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV). Other examples of ISVs include, without limitation, Pulmonary Vascular Resistance (PVR); Cardiac Output (CO); hemoglobin, and rate of hemoglobin production/loss.

The term “nominal” in reference to a datum for a patient means a value that is nominal for a population to which the patient belongs. For example, a patient to which the patient belongs may be defined as a population of patients of the same age, and/or a population of patients of the same gender.

The term “null” in reference to a datum for a patient means an empty measurement value. Substituting a null value for the value of an as-measured datum simulates a scenario in which the as-measured datum was not received by the system.

The term “patient state” means a qualitative description of the physiology of a patient at a particular point of time of the patient's clinical course, which qualitative description is derived from quantified evidence (e.g., measurements of one or more of the patient's internal state variables), and which qualitative description is recognizable by medical practice, and may have implications to clinical decision-making. A patient state may be a medical condition, such as an adverse medical condition, for example. The term “patient state” does not include the patient's state of consciousness (e.g., awake and/or asleep; etc.)

Examples of particular patient states include, but are not limited to, adverse medical conditions such as inadequate delivery of oxygen, inadequate ventilation of carbon dioxide, hyperlactatemia, acidosis; amongst others. In addition, these patient states may be specific to a particular medical condition, and the bounds of each of the patient states may be defined by threshold values of various physiological variables and data.

A “set” includes at least one member.

System Modules and Interaction

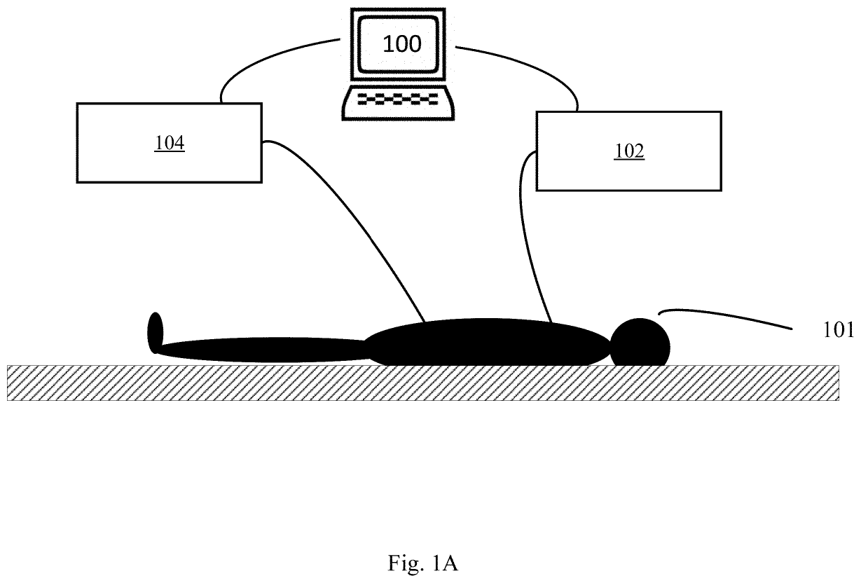

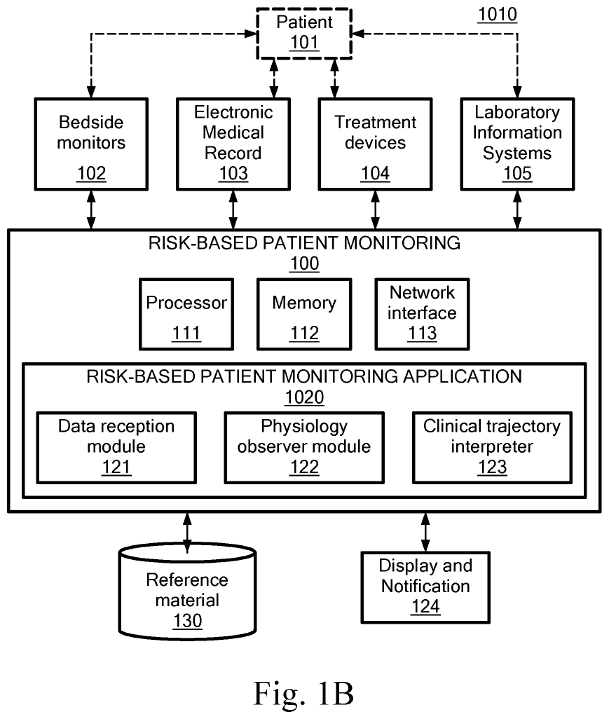

Referring now to the figures, FIG. 1A and FIG. 1B illustrate an embodiment of a medical care risk-based monitoring environment 1010 for providing health providers, such as physicians, nurses, or other medical care providers, risk-based monitoring in accordance with various embodiments of the present disclosure. A patient 101 may be coupled to one or more physiological sensors or bedside monitors 102 that may monitor various physiological parameters of the patient. It should be noted that a patient may be a human, or not human (a non-human being).

These physiological sensors may include but are not limited to, a blood oximeter, a blood pressure measurement device, a pulse measurement device, a glucose measuring device, one or more analyte measuring devices, an electrocardiogram recording device, amongst others. In addition, the patient may be administered routine exams and tests and the data stored in an electronic medical record (EMR) 103. The electronic medical record 103 may include but is not limited to stored information such as hemoglobin, arterial and venous oxygen content, lactic acid, weight, age, sex, ICD-9 code, capillary refill time, subjective clinician observations, patient self-evaluations, prescribed medications, medications regiments, genetics, etc. In addition, the patient 101 may be coupled to one or more treatment devices 104 that are configured to administer treatments to the patient. In some embodiments, one or more treatment devices 104 may be controlled by a system 100 as disclosed herein, for example in response to output defining a patient state or medical condition from a trajectory interpreter module. In various embodiments, the treatments devices 104 may include extracorporeal membrane oxygenator, ventilator, medication infusion pumps, etc.

By way of the present disclosure, the patient 101 may be afforded improved risk-based monitoring over existing methods. A patient specific risk-based monitoring system, generally referred to herein as system 100, may be configured to receive patient related information, including real-time information from bed-side monitors 102, EMR patient information from electronic medical record 103, information from treatment devices 104, such as settings, infusion rates, types of medications, and other patient related information, which may include the patient's medical history, previous treatment plans, results from previous and present lab work, allergy information, predispositions to various conditions, and any other information that may be deemed relevant to make an informed assessment of the possible patient conditions and states, and their associated probabilities. For the sake of simplicity, the various types of information listed above will generally be referred to hereinafter as “patient-specific information”. In addition, the system may be configured to utilize the received information, determine the clinical risks, which then can be presented to a medical care provider, including but not limited to a physician, nurse, or other type of clinician.

The system, in various embodiments, includes one or more of the following: a processor 111, a memory 112 coupled to the processor 111, and a network interface 113 configured to enable the system to communicate with other devices over a network. In addition, the system may include a risk-based monitoring application 1020 that may include computer-executable instructions, which when executed by the processor 111, cause the system to implement risk-based monitoring of the patients, such as the patient 101.

The risk-based monitoring application 1020 includes, for example, a data reception module 121, a physiology observer module 122, a clinical trajectory interpreter module 123 (or, in some embodiments, risk calculation engine 123), and a visualization and user interaction module 124. In an illustrative embodiment, the data reception module 121 may be configured to receive data from bedside monitors 102, electronic medical records 103, treatment devices 104, and any other information that may be deemed relevant to make an informed assessment regarding the patient's clinical risks, and any combination thereof of the preceding elements.

The physiology observer module 122 utilizes multiple measurements to estimate probability density functions (PDF) of internal state variables (ISVs), including internal state variables that describe the components of the physiology relevant to the patient treatment and condition in accordance with a predefined physiology model. The ISVs may be directly observable with noise (as a non-limiting example, heart rate is a directly observable ISV), hidden (as a non-limiting example, oxygen delivery (DO₂) defined as the flow of blood saturated oxygen through the aorta cannot be directly measured and is thus hidden), or measured intermittently (as a non-limiting example, hemoglobin concentration as measured from Complete Blood Count tests is an intermittently observable ISV). In some embodiments, when the physiology observer module 122 evaluates a set of ISVs at a given time step (e.g., tₖ; tₖ₊₁; generally tₖ₊ₙ), the system 100 may not have a complete set of ISV measurements contemporaneous with that given time step. For example, the system 100 may have measurements for that given time step for some internal state variables, but may not have measurements for that given time step for some other internal state variables (e.g., a contemporaneous measurement for an intermittent ISV may not be available for the given time step). Consequently, that intermittent ISV is, for purposes of evaluating ISVs at the given time step, a hidden ISV. However, evaluation of the set of ISVs by the physiology observer module 122 (as described herein) is nevertheless possible according to embodiments described herein because the predicted PDFs of ISVs 211 carry in them the influence of past measurements of that intermittent ISV, and consequently those predicted PDFs of ISVs 211 are, in illustrative embodiments, sufficient input for the physiology observer module 122.

In illustrative embodiments, instead of assuming that all variables can be estimated deterministically without error, the physiology observer module 122 of the present disclosure provides probability density functions as an output. Additional details related to the physiology observer module 122 are provided herein.

The clinical trajectory interpreter module 123 may be configured, for example, with multiple possible patient states, and may determine which of those patient states are probable and with what probability (i.e., the probability of the patient being in a given patient state may be referred-to as the risk that the patient is in the given patient state), given the estimated probability density functions of the internal state variables. Examples of particular patient states include, but are not limited to, hypotension with sinus tachycardia, hypoxia with myocardial depression, compensated circulatory shock, cardiac arrest, hemorrhage, amongst others. In addition, these patient states may be specific to a particular medical condition, and the bounds of each of the patient states may be defined by threshold values of various physiological variables and data. In various embodiments, the clinical trajectory interpreter module 123 may determine the patient conditions under which a patient may be categorized using any of information gathered from reference materials, information provided by health care providers, other sources of information. The reference materials may be stored in a database or other storage device 130 that is accessible to the risk-based monitoring application 1020 via network interface 113, for example. These reference materials may include material synthesized from reference books, medical literature, surveys of experts, physician provided information, and any other material that may be used as a reference for providing medical care to patients. In some embodiments, the clinical trajectory interpreter module 123 may first identify a patient population that is similar to the subject patient being monitored. By doing so, the clinical trajectory interpreter module 123 may be able to use relevant historical data based on the identified patient population to help determine the possible patient states.

The clinical trajectory interpreter module 123 is capable of also determining the probable patient states under which the patient can be currently categorized, given the estimated probability density functions of the internal state variables, as provided by physiology observer module 122. In this way, each of the possible patient states is assigned a probability value from 0 to 1. The combination of patient states and their probabilities is defined as the clinical risk to the patient. Additional details related to the clinical trajectory interpreter module 123 are provided herein.

Visualization and user interactions module 124 may be equipped to take the outputs of the data reception module 121 the physiology observer module 122, and the clinical trajectory interpreter module 123 and present them to the clinical personnel. The visualization and user interactions module 124 may show the current patient risks, their evolution through time, the probability density functions of the internal state variables as functions of time, and other features that are calculated by the two modules 122 and 123 as by-products and are informative to medical practice. Additionally, visualization and user interactions module 124 enables the users to set alarms based on the patient state probabilities, share those alarms with other users, take notes related to the patient risks and share those notes with other users, and browse other elements of the patient medical history. Additional details related to the visualization and user interactions module 124 are provided herein.

I. Physiology Observer Module 122

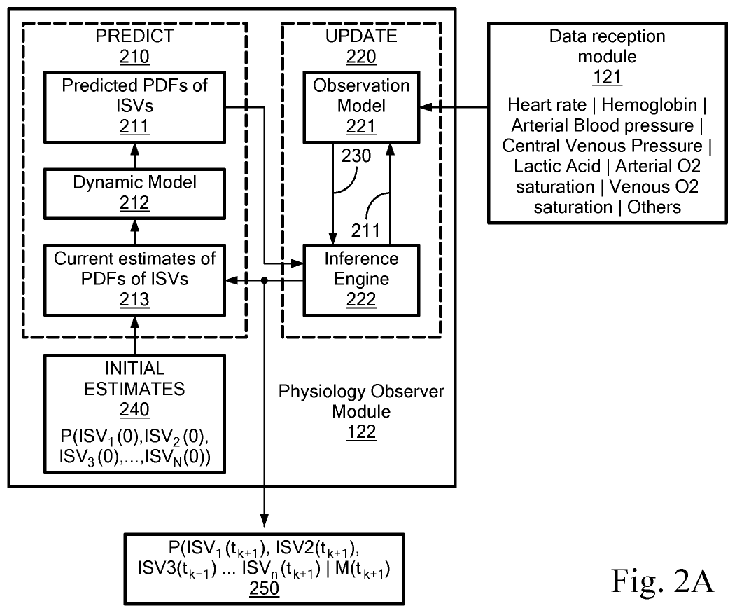

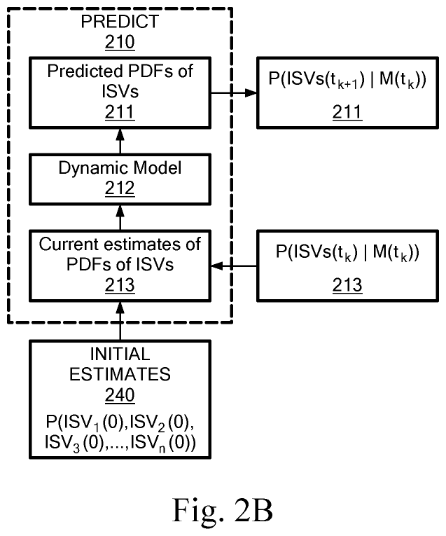

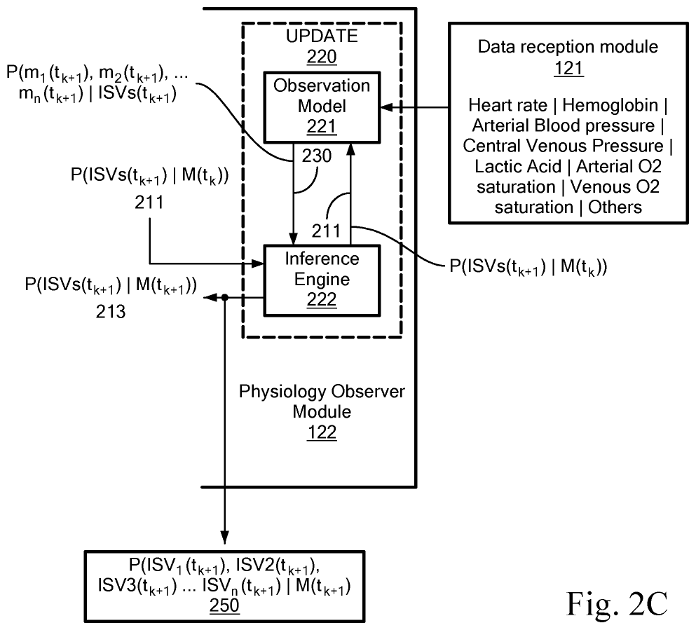

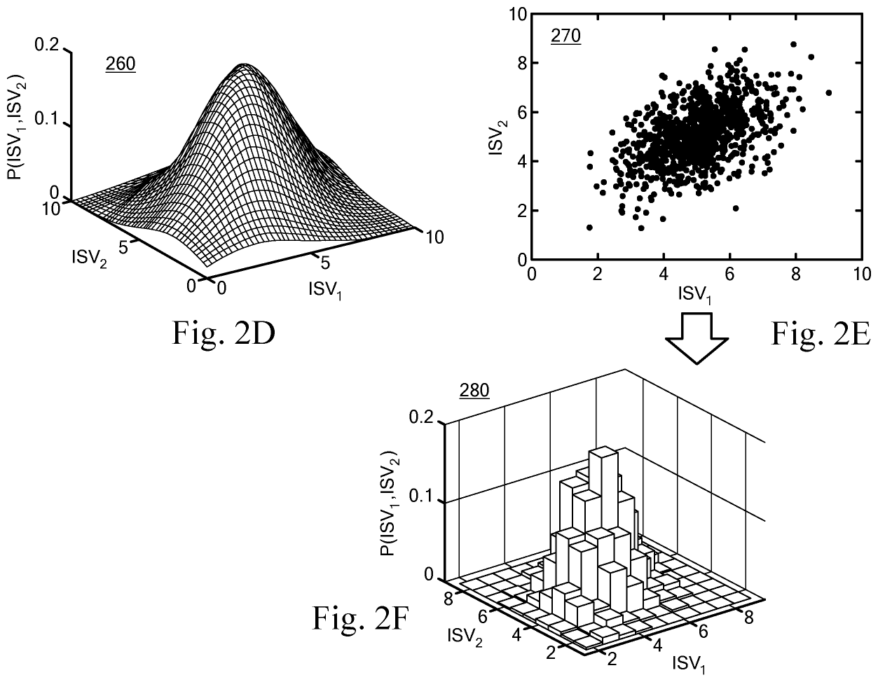

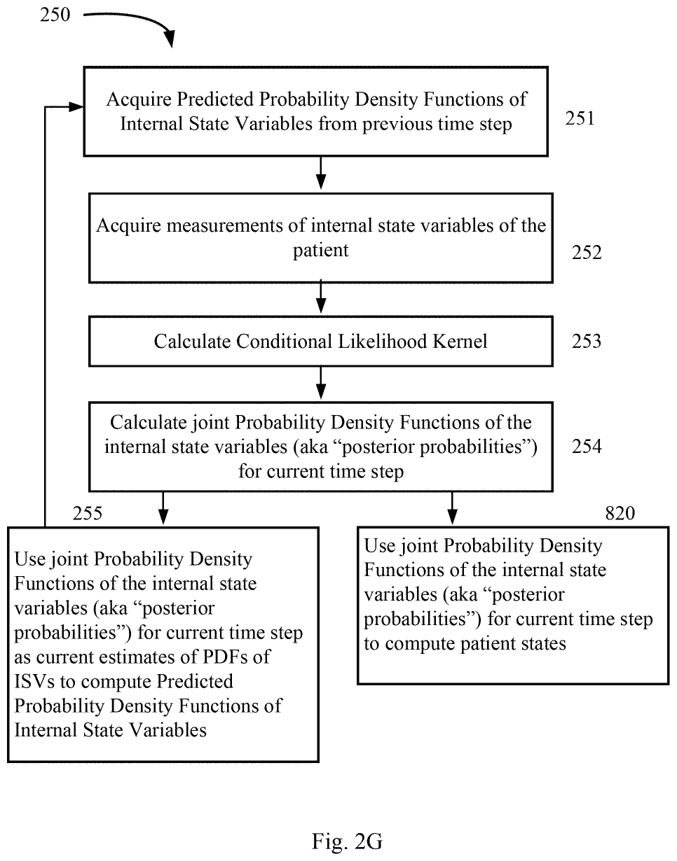

FIG. 2A illustrates a basic schematic of the physiology observer module 122, which utilizes two models of the patient physiology: a dynamic model (or dynamic module) 212 and an observation model (or observation module) 221. The dynamic model 212 captures the relationship arising between the internal state variables at some time tₖ and another close, subsequent time tₖ₊₁, thereby enabling modeling of the patient physiology as a system whose present state has information about the possible future evolutions of the system. Given the propensity of the patient physiology to remain at homeostasis through auto-regulation, and the physical laws guiding different processes in the human body, e.g. fluid mechanics, chemical reactions; there is a clear rationale of introducing dynamic equations that capture the evolution of the system from a present state to a future state.

The observation model 221 may capture the relationships between measured physiology variables and other internal state variables. Examples of such models include: a) the dependence of the difference between systolic and diastolic arterial blood pressures (also called pulse pressure) on the stroke volume; b) the relationship between measured heart rate and actual heart rate; c) the relationship between pulse oximetry and arterial oxygen saturation; and d) any other dependence between measurable and therefore observable parameters and internal state variables.

The physiology observer module 122 functions as a recursive filter by employing information from previous measurements to generate predictions of the internal state variables and the likelihood of probable subsequent measurements (i.e., future measurements, relative to the previous measurements) and then comparing them with subsequent measurements (e.g., the most recently acquired measurements). Specifically, the physiology observer module 122 utilizes the dynamic model 212 in the predict step or mode 210 and the observation model 221 in the update step or mode 220. In the following illustrative example, operation of the physiology observer module 122 over successive time steps is described using FIG. 2B and FIG. 2C. For purposes of illustration in those figures, the previous time step will be denoted tₖ, the subsequent time step will be denoted tₖ₊₁. It should be noted that the previous time step tₖ, itself was preceded by an earlier time step tₖ₋₁. Consequently, the time steps in this illustrative embodiment proceed from tₖ₋₁ to tₖ to tₖ₊₁.

A. Predict Module 210 {FIG. 2B}

During the prediction mode 210, at or after time step tₖ and on or before time step tₖ₊₁, the physiology observer module 122 takes the estimated probability density functions (PDFs) of ISVs 213 at a time step tₖ (which were produced at time step tₖ based in part from data from earlier time step tₖ₋₁, and which may be referred-to as the posterior probabilities for time step tₖ) and feeds them to the dynamic model 212, which produces predictions of the probability density functions of the ISVs 211 for the next time step tₖ₊₁.

This is accomplished using the following equation:

P(ISVs(tₖ₊₁)|M(tₖ))=∫_{ISVs∈ISV}P(ISVs(tₖ₊₁)|ISVs(tₖ))P(ISVs(tₖ)|M(tₖ))dISVs

Where:

ISVs(tₖ)={ISV₁(tₖ),ISV₂(tk),ISV₃(tₖ), . . . ISVₙ(tₖ)}; and

M(tₖ) is the set of all measurements up to time tₖ.

The probability P(ISVs(tₖ₊₁)|ISVs(tₖ)) defines a transition probability kernel describing the dynamic model 212, which defines how the estimated PDFs evolve with time.

The probabilities P(ISVs(tₖ)|M(tₖ)) are provided by the inference engine 222 and are the posterior probabilities of the ISVs given the measurements acquired at the earlier time step tₖ₋₁.

B. Update Module 220

During the update mode 220 of the physiology observer module 122, the predicted probability density functions of the ISVs 211 (i.e., which were produced using the predict module 210 and the density function at the preceding time step tₖ) are compared against the measurements received (at time tₖ₊₁) from data reception module 121 with the help of the observation model 221, and as a result the ISVs are updated to reflect the new available information. Processes of the Observation Model 221 are described in more detail, below.

1. Observation Model {221}

The observation model 221 produces a conditional likelihood kernel 230 [for example: [P(m₁(tₖ₊₁), m₂(tₖ₊₁), . . . mₙ(tₖ₊₁)|ISVs(tₖ₊₁))] and provides the conditional likelihood kernel 230 to the Inference Engine 222. The conditional likelihood kernel 230 provided by the observation model 221 determines how likely the currently received measurements are given the currently predicted ISVs (i.e., the predicted PDFs of ISVs produced by the predict module 210 at the immediately previous time step). Criteria for determining how likely the currently received measurements are, given the currently predicted ISVs, may be established by the discretion of the system's designer, based on the particular application faced by the designer.

Note that the observation model 221 has two inputs and one output. The inputs are (1) the received measurements from data reception module 121, and (2) the predicted probability density functions of the ISVs 211 [e.g., P(ISVs(tk+1)|M(tk))], provided via the Inference Engine 222, represented by the arrow pointing from the Inference Engine 222 to the Observation Module 221. The output of the observation model 221 is the conditional likelihood kernel 230.

2. Operation of the Observation Model {221}

The processes of the Observation Model 221 are described below.

Note that the measurements received at the Data Reception Module 121 are individual quantitative measurements at discrete points in time, but subsequent processing steps (e.g. Bayes Theorem at the Inference Engine 222, and the operation of the Clinical Trajectory Interpreter Module 123) operate on PDFs (probability density functions) as inputs, rather than such discrete data points. Consequently, one of the functions of the Observation Model 221 is to intake the discrete quantitative measurements of internal state variables and output PDFs. This functionality is explained below.

(a). Comparing the Received Measurements to the Predicted PDFs of ISVs 211

During the update mode 220 of the physiology observer module 122, the predicted PDFs of ISVs 211 (which were generated using the predict module and the PDFs of the ISVs from preceding time step tₖ) are compared against the measurements received at the subsequent time step (tₖ₊₁) from data reception module 121 with the help of the observation model 221, and as a result the ISVs are updated to reflect the new available information.

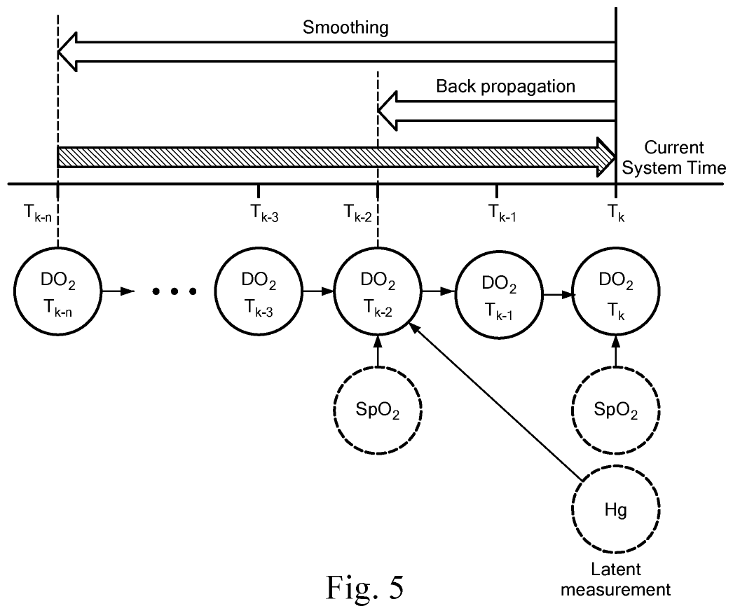

As mentioned above, certain measurements, such as Hemoglobin, are available to the system with an unknown amount of time latency, meaning the measurements are valid in the past relative to the current time and the time they arrive over the data communication links. The physiology observer module 122 may handle such out of sequence measurements using back propagation, in which the current estimates of the ISVs are projected back in time to the time of validity of the measurements, so that the information from the latent measurement can be incorporated correctly. FIG. 5 depicts such time line. In FIG. 5, hemoglobin arrives at the current system time, tₖ, but is valid and associated back to the ISV (DO2) at time Tₖ₋. Back propagation is the method of updating the current ISVs probability estimates P(ISVs(tₖ)|M(tₖ)) with a measurement that is latent relative to the current time, m(tₖ₋ₙ). Back propagation is accomplished in a similar manner to the prediction method described previously. There is a transition probability kernel, P(ISVs(tₖ₋ₙ)|ISVs(tₖ)), that defines how the current probabilities evolve backwards in time. This can then be used to compute probabilities of the ISVs at time tₖ₋ₙ given the current set of measurements which excludes the latent measurement, as follows:

P(ISVs(tₖ₋ₙ)|M(tₖ))=∫_{ISVs∈ISV}P(ISVs(tₖ₋ₙ)|ISVs(tₖ))P(ISVs(tₖ)|M(tₖ))dISVs

Once these probabilities are computed, the latent measurement information is incorporated using Bayes' rule in the standard update:

P(ISVs(tₖ₋ₙ)|M(tₖ),m(tₖ₋ₙ))=P(M(tₖ₋ₙ)|ISVs(tₖ₋ₙ))P(ISVs(tₖ₋ₙ)|M(tₖ)/P(M(tₖ),m(tₖ₋ₙ))

The updated probabilities are then propagated back to the current time tₖ using the prediction step described earlier. Back propagation can be used to incorporate the information.

Another functionality of the physiology observer module 122 includes smoothing. The care provider using the system 100 may be interested in the patient state at some past time. With smoothing, the physiology observer module 122 may provide a more accurate estimate of the patient ISVs at that time in the past by incorporating all of the new measurements that the system has received since that time, consequently providing a better estimate than the original filtered estimate of the overall patient state at that time to the user, computing P(ISVs(tₖ₋ₙ)|M(tₖ)). This is accomplished using the first step of back propagation in which the probability estimates at time tₖ which incorporate all measurements up to that time are evolved backwards to the time of interest tₖ₋ₙ using the defined transition probability kernel. This is also depicted in FIG. 5, in which the user is interested in the patient state at tₖ₋ₙ and the estimates are smoothed back to that time.

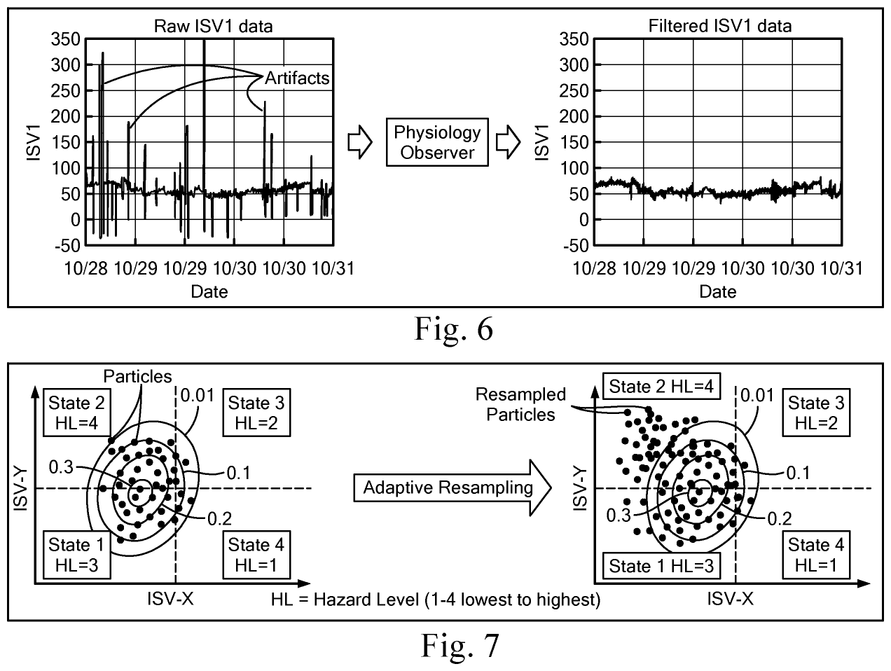

In addition, because physiology observer module 122 maintains estimates of each of the measurements available to the system 100 based on physiologic and statistical models, module 122 may filter artifacts of the measurements that are unrelated to the actual information contained in the measurements. This is performed by comparing the newly acquired measurements with the predicted likelihoods of probable measurements given the previous measurements. If the new measurements are considered highly unlikely by the model, they are not incorporated in the estimation. The process of comparing the measurements with their predicted likelihoods effectively filters artifacts and reduces noise. FIG. 6 shows an example of such a process involving mean arterial blood pressure (ABPm). FIG. 6 shows the raw ABPm measurements prior to being processed by the physiology observer module 122 with the measurement artifacts identified, as well as the filtered measurements after being processed by the physiology observer module 122. As can be seen, the measurement artifacts have been removed and the true signal is left.

(b). Creating the Conditional Likelihood Kernel

The “Conditional Likelihood Kernel” 230 [P(m₁(tₖ₊₁), mₙ(tₖ₊₁), . . . mₙ(tₖ₊₁)|ISVs(tₖ₊₁))] determines how likely the currently received measurements are given the currently predicted ISVs. As can be seen from the foregoing formula, the Conditional Likelihood Kernel 230 includes a set of probability density functions of the measurements {mₙ at time tₖ₊₁} assuming (or based on) the ISVs predicted for time step tₖ₊₁ (i.e., the predicted PDFs of ISVs 211 for time step tₖ₊₁, which were generated at time step tₖ). Note that from this point forward, the algorithms no longer operate on the discrete measurements from the data reception module 121 per se.

In general, creating probably density functions of ISVs (i.e., the components of the Conditional Likelihood Kernel 230) is performed by an “inference scheme.” There are several such inference schemes, including for example exact inference schemes.

In various embodiments, physiology observer module 122 may utilize a number of algorithms for estimation or inference. Depending on the physiology model used, the physiology observer module 122 may use exact inference schemes, such as the Junction Tree algorithm, or approximate inference schemes using Monte Carlo sampling such as a particle filter, or a Gaussian approximation algorithms such as a Kalman Filter or any of its variants.

As discussed, the physiology model used by physiology observer module 122 may be implemented using a probabilistic framework known as a Dynamic Bayesian Network, which graphically captures the causal and probabilistic relationship between the ISVs of the system, both at a single instance of time and over time. Because of the flexibility this type of model representation affords, the physiology observer module 122 may utilize a number of different inference algorithms. The choice of algorithm is dependent on the specifics of the physiology model used, the accuracy of the inference required by the application, and the computational resources available to the system. Used in this case, accuracy refers to whether or not an exact or approximate inference scheme is used. If the physiology observer model is of limited complexity, then an exact inference algorithm may be feasible to use. In other cases, for more complex physiology observer models, no closed form inference solution exists, or if one does exist, it is not computationally tractable given the available resources. In this case, an approximate inference scheme may be used.

The simplest case in which exact inference may be used, is when all of the ISVs in the physiology model are continuous variables, and relationships between the ISVs in the model are restricted to linear Gaussian relationships. In this case, a standard Kalman Filter algorithm can be used to perform the inference. With such algorithm, the probability density function over the ISVs is a multivariate Gaussian distribution and is represented with a mean and covariance matrix.

When all of the ISV's in the model are discrete variables, and the structure of the graph is restricted to a chain or tree, the physiology observer module 122 may use either a Forward-backward algorithm, or a Belief Propagation algorithm for inference, respectively. The Junction Tree algorithm is a generalization of these two algorithms that can be used regardless of the underlying graph structure, and so the physiology observer module 122 may also use this algorithm for inference. Junction Tree algorithm comes with additional computational costs that may not be acceptable for the application. In the case of discrete variables, the probability distribution functions can be represented in a tabular form. It should be noted that in the case where the model consists of only continuous variables with linear Gaussian relationships, these algorithms may also be used for inference, but since it can be shown that in this case these algorithms are equivalent to the Kalman Filter, the Kalman Filter is used as the example algorithm.

When the physiology model consists of both continuous and discrete ISVs with nonlinear relationships between the variables, no exact inference solution is possible. In this case, the physiology observer module 122 may use an approximate inference scheme that relies on sampling techniques. The simplest version of this type of algorithm is a Particle Filter algorithm, which uses Sequential Importance Sampling. Markov Chain Monte Carlo (MCMC) Sampling methods may also be used for more efficient sampling. Given complex and non-linear physiologic relationships, this type of approximate inference scheme affords the most flexibility. A person reasonably skilled in the relevant arts will recognize that the model and the inference schemes employed by the physiology observer module may be any combination of the above described or include other equivalent modeling and inference techniques.

When using particle filtering methods, a resampling scheme is desirable to avoid particle degeneracy. The physiology observer may utilize an adaptive resampling scheme. As described in detail herein, regions of the ISV state space may be associated with different patient states, and different levels of hazard to the patient. The higher the number, the more hazardous that particular condition is to the patient's health. In order to ensure accurate estimation of the probability of a particular patient condition, it may be necessary to have sufficient number of sampled particles in the region. It may be most important to maintain accurate estimates of the probability of regions with high hazard level and so the adaptive resampling approach guarantees sufficient particles will be sampled in high hazard regions of the state space. FIG. 7 illustrates an example of this resampling based on two internal state variables (“ISV-X” and “ISV-Y”), which may be any internal state variables of the patient, including without limitation any of the internal state variables described herein. State 1 and State 2 have the highest hazard level. The left plot depicts the samples generated from the standard resampling. Notice there are naturally more particles in state 1 and state 2 region because these states are most probable. The right plot shows the impact of the adaptive resampling. Notice how the number of samples in the areas of highest risk has increased significantly.

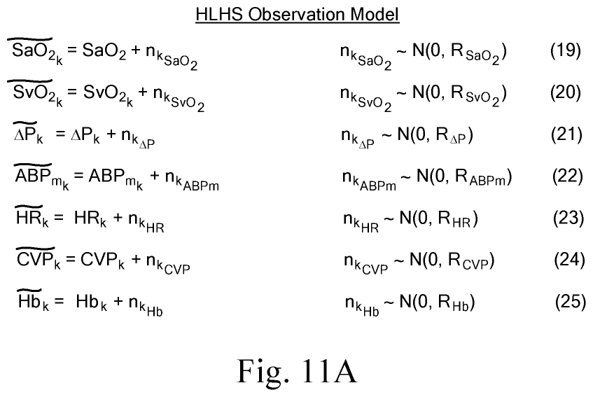

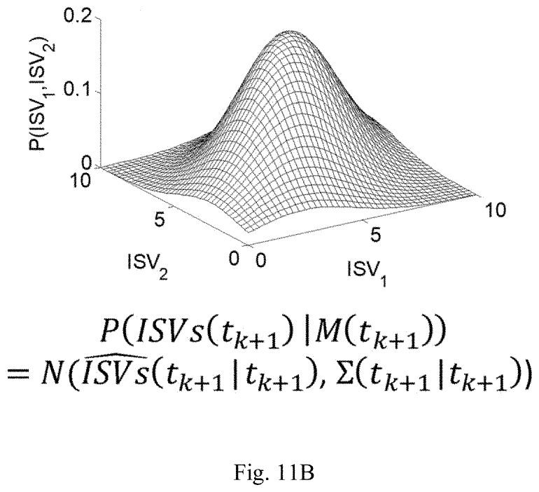

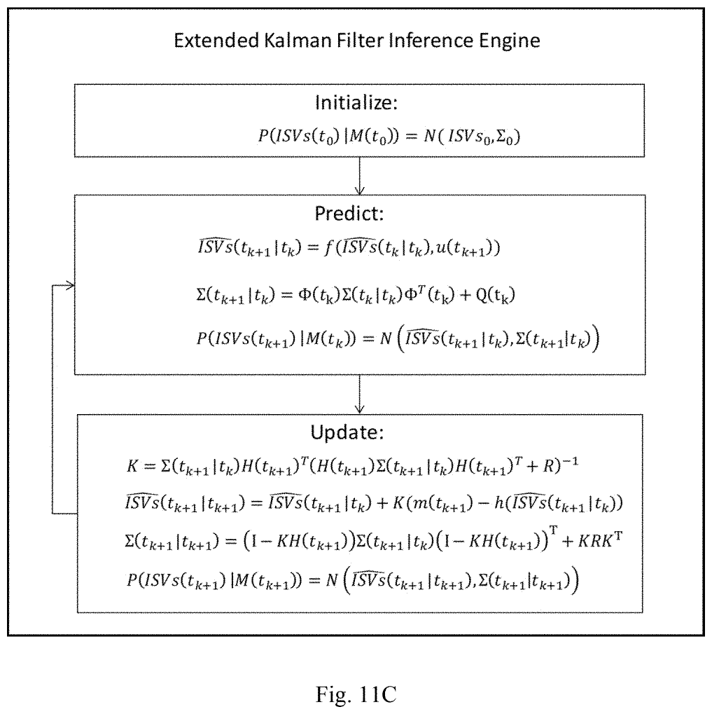

FIG. 11A, described further below, illustrate an example of creating the Conditional Likelihood Kernel 230 in the context of “HLHS Stage 1” example, (where “HLHS” is Hypoplastic Left Heart Syndrome”).

As described in connection with FIG. 11A, in the observation model 221 inference over the DBN is performed using an Extended Kalman Filter (“EKF”), which is a variant of the Kalman Filter that extends the inference engine algorithm for use on applications where the underlying models have nonlinear relationships. The extension is accomplished using a Taylor-series expansion of the nonlinear relationships of the model. This approximation allows the algorithm analytically calculate a Gaussian approximation to the posterior density given the measurements provided to the system.

FIG. 11B depicts this Gaussian approximation for P(ISVs(tₖ₊₁)|M(tₖ₊₁)). The depicted density is a multivariate Gaussian that can be fully represented using the conditional mean of the ISVs at the current time, [symbol] (tₖ₊₁|tₖ₊₁), and the conditional covariance matrix of the ISVs, Σ(tₖ₊₁|tₖ₊₁). These quantities are conditioned on all of the available measurements up to the current time step.

FIG. 11C, depicts the computation steps of the EKF as they relate to the current implementation. In the first step, the density is initialized using assumed initial mean and covariance conditions for the ISVs which can be informed by patient population norms or medical literature. After initialization, the density is passed to the predict step in which the ISV density is predicted forward to the time of the current measurement. This prediction is accomplished by predicting the conditional mean forward in time using the dynamic model specified in the physiology observer module, and by predicting the conditional covariance matrix utilizing a linearization of this dynamic model with the addition of “process noise” to account for uncertainty in the model of the dynamics. Note, this is the same calculation that is described in the predict module of physiology observer module except specifically for Gaussian densities. Because the EKF algorithm has a predict step incorporated in the calculation, the physiology observer is able to utilize this predict method as part of the processing.

It should be noted that the Extended Kalman Filter, as described above, is not limited to use in creating the Conditional Likelihood Kernel 230 in the context of “HLHS Stage 1.” Rather, the Extended Kalman Filter may be used to create any Conditional Likelihood Kernel 230 supported by this disclosure.

Following the prediction of the PDF to the current measurement time, the posterior density is calculated using Bayes Rule combining the new information provided by the current measurement m(tₖ₊₁) with the prior predicted PDF in the update step. Because of the Gaussian approximation, this calculation is analytically tractable and only involves calculating the posterior conditional mean and posterior conditional covariance matrix. Once these quantities have been updated, the entire density can be calculated.

The update step takes as an input the observation model specified in the physiology observer. Using this model, the calculation first calculates the Kalman Gain (K) which determines how much the posterior conditional mean and covariance matrix change from prior given on the new data. The Kalman Gain is a function of the prior conditional covariance matrix, the expected noise associated with the measurement and a linearization of the observation model provided by the observer. Once the Kalman Gain is computed, the posterior conditional mean is updated from the prior mean using the difference between the measurement value and the expected measured value scaled by this gain. The posterior conditional covariance is updated from the prior in a similar manner, reducing the overall uncertainty proportional to the amount of information that the measurement provides about the underlying ISVs. Following this step, the conditional density is passed back to the predict method 210 where it is predicted to the next (i.e., subsequent) measurement step. It is also returned to the physiology observer module.

3. Inference Engine {222}

The inference engine 222 of physiology observer module 122 achieves this update by using the predicted probability density functions of the ISVs 211 as a-priori probabilities, which are updated with the statistics (i.e., the conditional likelihood kernel 230) of the received measurements from data reception module 121 to achieve the posterior probabilities reflecting the current (at time tₖ₊₁) probability density functions of the ISVs 213. The inference engine 222 accomplishes the update step 220 with the following equation which is Bayes' Theorem,

$$P ( ISV s ( {t}_{k + 1} ) \vert M ( {t}_{k + 1} ) ) = \frac{P ( \begin{matrix}{m}_{1} ( {t}_{k + 1} ) , {m}_{2} ( {t}_{k + 1} ) , … \\ {m}_{n} ( {t}_{k + 1} ) \vert ISVs ( {t}_{k + 1} )\end{matrix} ) P ( ISVs ( {t}_{k + 1} ) \vert M ( {t}_{k} ) )}{P ( {m}_{1} ( {L}_{k + 1} ) , {m}_{2} ( {L}_{k + 1} ) , … {m}_{n} ( {L}_{k + 1} ) \vert M ( {t}_{k} ) )}$$

where:

P(ISVs(tₖ₊₁)|M(tₖ₊₁)) are the “posterior probabilities” 250 at time step tₖ₊₁ expressed as conditional probabilities;

P(m₁(tₖ₊₁), m₂(tₖ₊₁), . . . mₙ(tₖ₊₁)|ISVs(tₖ₊₁)) is the conditional likelihood kernel provided by the observation model 221 that determines how likely the currently received measurements are given the currently predicted ISVs (i.e., the predicted PDFs of ISVs 211 created for previous time step tₖ);

P(ISVs(tₖ₊₁)|M(tₖ)) are the predicted PDFs of ISVs 211 produced in the predict model at the previous time step tₖ (see, e.g., FIG. 2B); and

P(m₁(tₖ₊₁), m₂(tₖ₊₁), . . . mₙ(tₖ₊₁)|M(tₖ)) is the predicted PDFs of the measurements received at the time step given the measurements received up to that time step.

At the initialization time (e.g., t=0 or t=tᵢₙᵢₜ) when no then-current estimate of probability density functions of the ISVs is available, the physiology observer module 122 may utilize initial estimates 240, which may be derived from an educated guess of possible values for the ISVs or statistical analysis of previously collected patient data.

Referring now to FIG. 2A, it can be seen that the output of the Inference Engine 222 at time step tₖ₊₁ (i.e., the “posterior probabilities” of time step tₖ₊₁) is sent in two directions.

In the first direction, the output of the Inference Engine 222 is provided to the predict module 210, where it may be referred-to as the “current estimates of PDFs of ISVs” 213 (see, e.g., FIG. 2B and its related description).

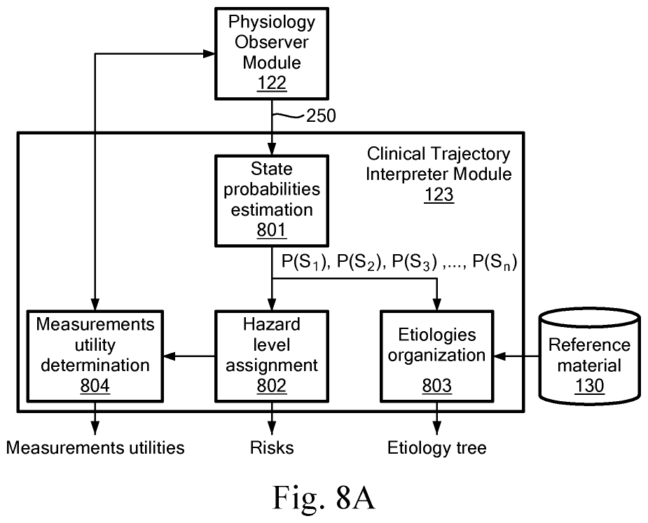

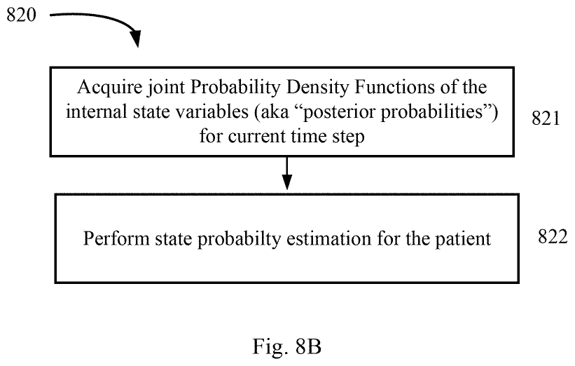

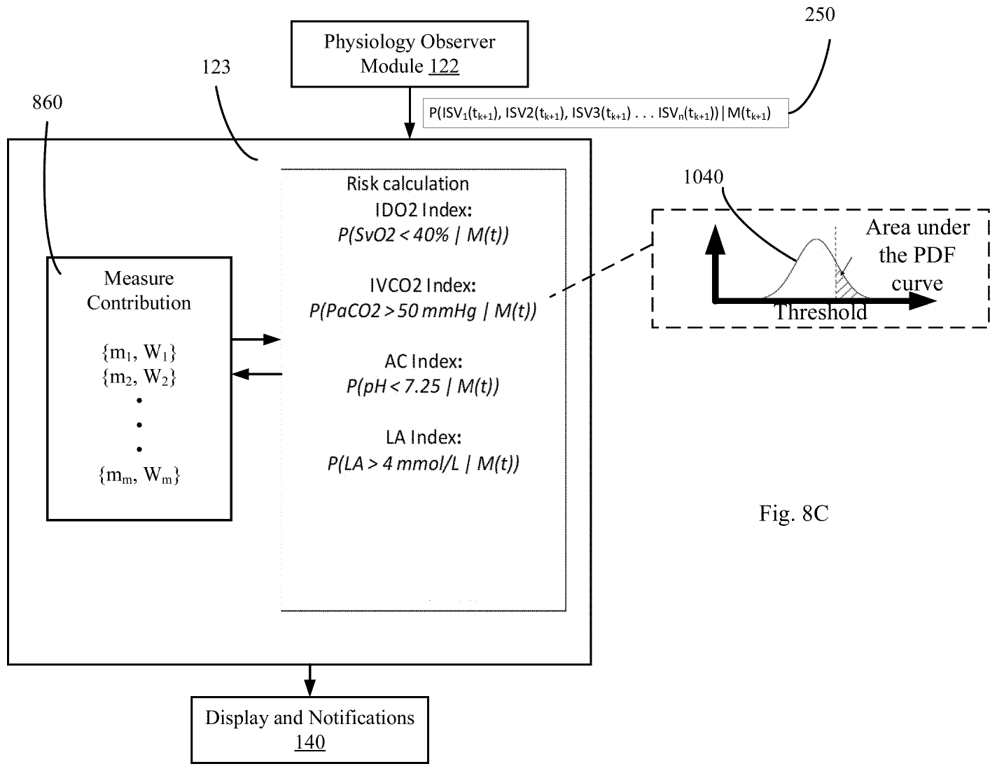

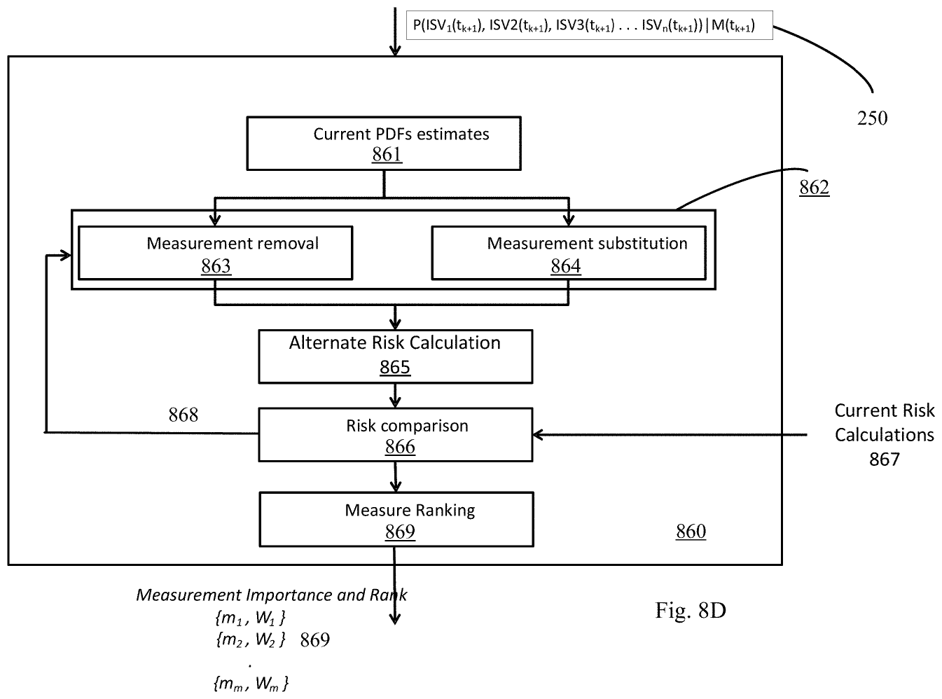

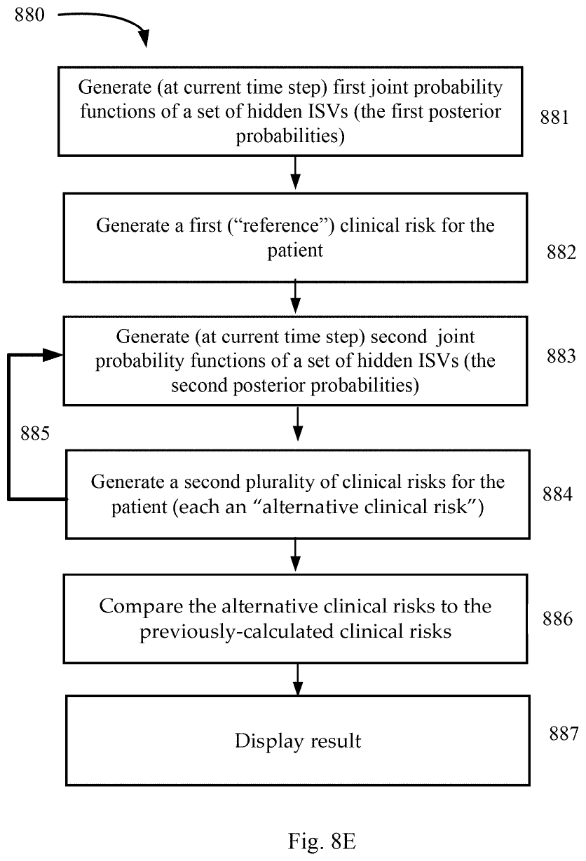

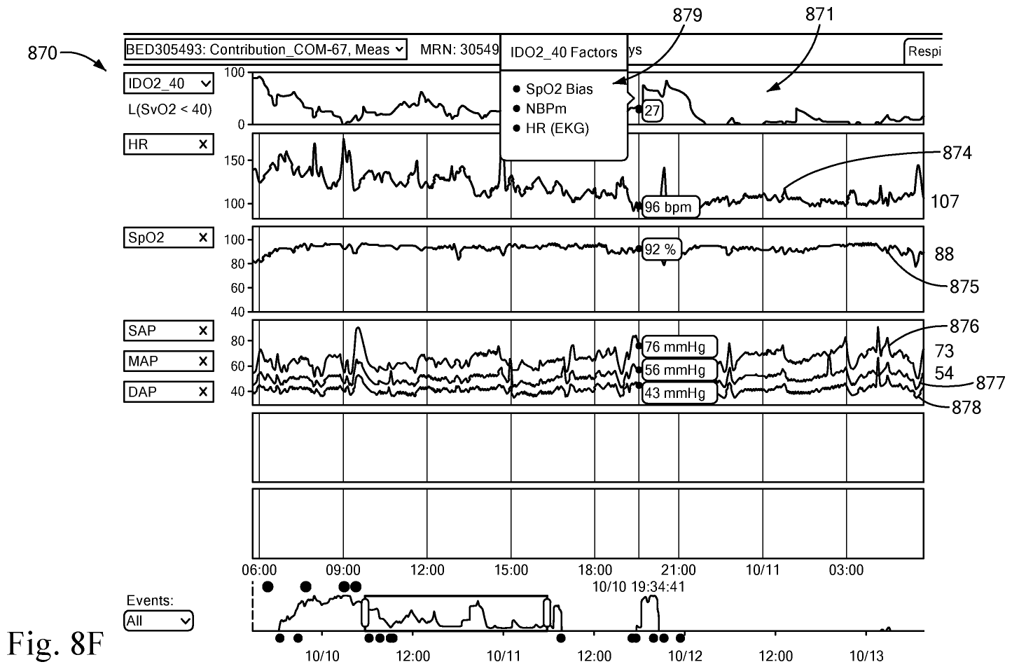

In the second direction, the output of the Inference Engine 222 at time step tₖ₊₁ (expressed in the figures simply as probabilities) is provided to the to the clinical trajectory interpreter module 123, where that output may be referred-to as the “joint Probability Density Functions of the ISVs from the physiology observer module”, and/or “posterior probabilities”, 250. Operation of the clinical trajectory interpreter module 123 is described further herein (see, e.g., FIG. 2C and its related description; and e.g., FIG. 8A).

II. Clinical Trajectory Interpreter Module {123} {Determining Patient States}

Using the posterior probabilities (250) from the Inference Engine 222 for time step tₖ₊₁, the Clinical Trajectory Interpreter Module 123 performs state probability estimation 801 to calculate the probabilities (i.e., risks) of one or more different patient states.

Referring now to FIG. 8A, the Clinical Trajectory Interpreter 123 takes the joint Probability Density Functions of the ISVs 250 from physiology observer module 122, and performs state probability estimation 801 to calculate the probabilities of one or more patient states.

FIG. 8B is a flowchart illustrating at a high level a method 820 of operation of the Clinical Trajectory Interpreter 123. At step 821, the Clinical Trajectory Interpreter 123 obtains or receives the joint probability density functions (also known as the “posterior possibilities”) produced by the Physiology Observer Module 122, and at step 822, the Clinical Trajectory Interpreter 123 performs state probability estimation of one or more patient states for the patient. The estimated probability for each such patient state may be referred to as the risk that the patient is in said patient state.

The joint Probability Density Functions of the ISVs 250 may be defined in closed form, for example multidimensional Gaussians 260, or approximated by histogram 280 of particles 270, as illustrated in FIG. 2D, FIG. 2E and FIG. 2F. In both cases, the probability density functions of the ISVs can be referred to as: (ISV1(t), ISV2(t), . . . , ISVn(t)), where t is the time they refer to.

Determining the patient states (i.e., “determining the probability of the patient being in a particular state Sᵢ”) may be done in a variety of ways.

Generally, the PDFs of the ISVs define a domain. In illustrative embodiments, the domain is partitioned into quadrants, each quadrant representing a patient state. The probability that the patient is in a given one of the four patient states is determined by the quantity of the PDFs of the ISV's located within the given quadrant.

This may be described as:
(i) partitioning a domain spanned by the internal state variables into different regions, each region defining a separate patient state; and
(ii) integrating the probability density functions over the regions corresponding to each particular patient state to produce probabilities that the patient may be classified under each of said possible patient states.

FIG. 2D: Multidimensional Gaussian 260

Where the data is in the form of a multidimensional Gaussian 260, integration may be performed directly:

P(Sᵢ(t))=∫_{-∞}^{∞} . . . ∫_{-∞}^{∞}P(S|ISV₁,ISV₂, . . . ,ISVₙ)P(ISV₁(t),ISV₂(t), . . . ,ISVₙ(t))dISV₁ . . . dISVₙ

FIG. 2E and FIG. 2F: Histogram of Particles 270

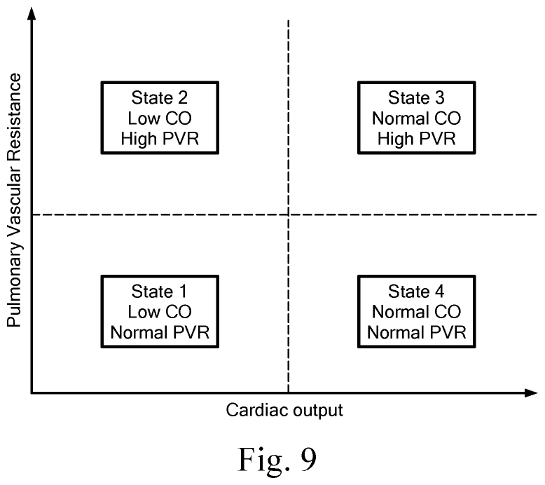

In case that the output 250 of the Inference Engine 222 is approximated by a histogram 280 of particles 270 and P(S|ISV₁, ISV₂, . . . , ISVₙ) is defined by a partition of the space spanned by ISV₁, ISV₂, . . . , ISVₙ into regions as shown in FIG. 9, the probability P(Sᵢ(t)) may be calculated by calculating the fraction of particles 270 in each region.

FIG. 2G is a flowchart describing an embodiment of a method 250 of operation of a Physiology Observer Module 122 at time step 2ₖ₊₁ in accordance with an illustrative embodiment.

Step 251 includes acquiring predicted probability density functions (211) of internal state variables produced at a previous time step (tₖ) for use by the Physiology Observer Module 122 at time step tₖ₊₁.

Step 252 includes acquiring measurement of internal state variables at time step tₖ₊₁, by the Data Reception Module 121.

Step 253 includes generating a Conditional Likelihood Kernel by the Observation Module 221.

Step 254 includes generating joint Probability Density Functions of the internal state variables (also referred-to as “posterior probabilities”), by the Inference Engine 222.

Step 255 includes using the posterior probabilities 250 as current (i.e., in this example, time step tₖ₊₁) estimates 213 of probability density functions of the internal state variables to compute Predicted Probability Density Functions of Internal State Variables (211) for use in a subsequent time step (tₖ₊₂).

FIG. 2G also includes step 820, which is further described in FIG. 8B.

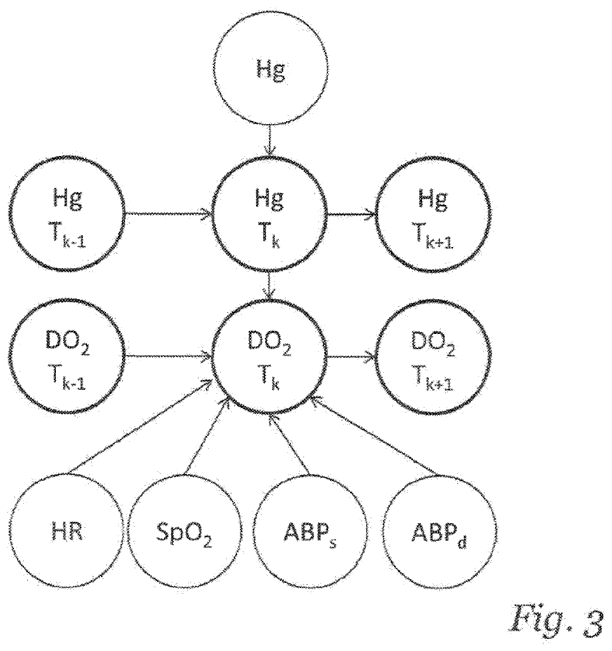

FIG. 3 illustrates a non-limiting example of models that enable the physiology observer in accordance with the present disclosure. While not directly observable, the management of oxygen delivery, DO2, is an important part of critical care. Therefore, precise estimation of DO2 can inform improved clinical practice. In the illustrated example, this estimation is achieved through the measurements of hemoglobin concentration (Hg), heart rate (HR), diastolic and systolic arterial blood pressures, and SpO2. The dynamic model 212 assumes that oxygen delivery is driven by a feedback process which stabilizes it against stochastic disturbances. Similarly, hemoglobin concentration is controlled around the norm value of 15 mg/dL. The observation model 221 takes into account the relationship between arterial oxygen saturation SpO2, hemoglobin concentration and arterial oxygen content CaO2, the dependence of the difference between systolic, ABPs, and diastolic, ABPd, arterial blood pressures (also called pulse pressure) on the stroke volume, and the relationship between heart rate, HR, stroke volume, SV, and cardiac output. The two models are abstracted as a Dynamic Bayesian Network (DBN), and the physiology observer module 122 utilizes the DBN to continuously track the oxygen delivery. A Dynamic Bayesian Network is a systematic way to represent statistical dependencies in terms of a graph whose vertices signify variables (observable and unobservable), and whose edges show causal relationships. Further descriptions of an exemplary DBN for DO2 estimation can be found in U.S. Provisional Application No. 61/699,492, filed on Sep. 11, 2012, entitled SYSTEMS AND METHODS FOR EVALUATING CLINICAL TRAJECTORIES AND TREATMENT STRATEGIES FOR OUTPATIENT CARE, Attorney Docket No. 3816/10301, and U.S. Provisional Application No. 61/684,241, filed on Aug. 17, 2012, entitled SYSTEM AND METHODS FOR PROVIDING RISK ASSESSMENT IN ASSISTING CLINICIANS WITH EFFICIENT AND EFFECTIVE BLOOD MANAGEMENT, Attorney Docket No. 3816/10101, to which priority is claimed, the disclosure of which is incorporated herein by reference.

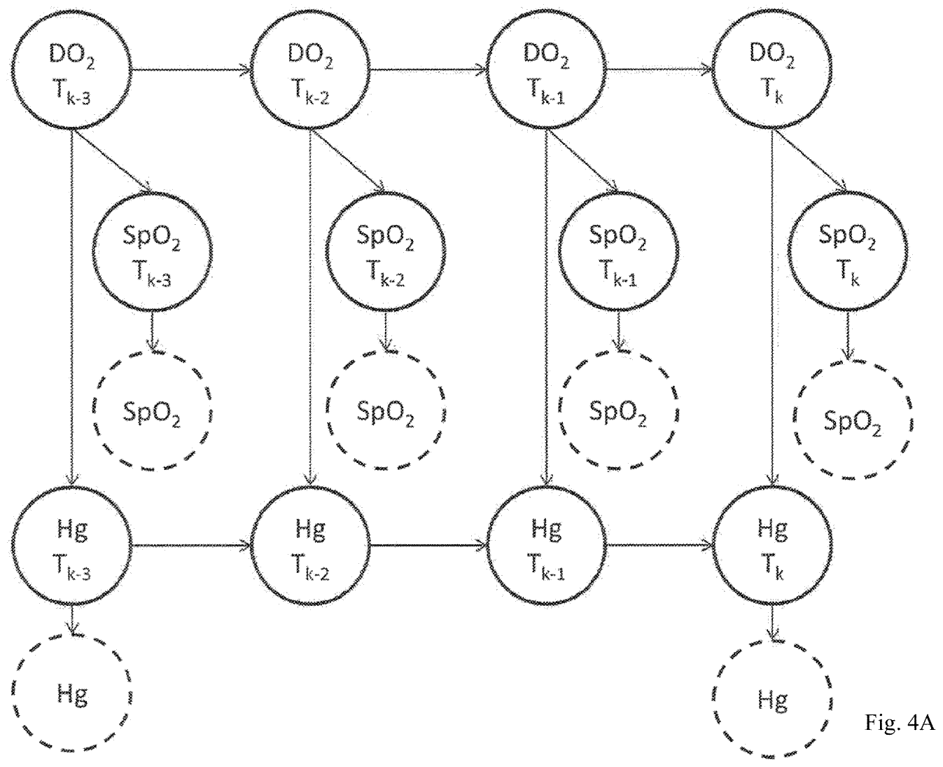

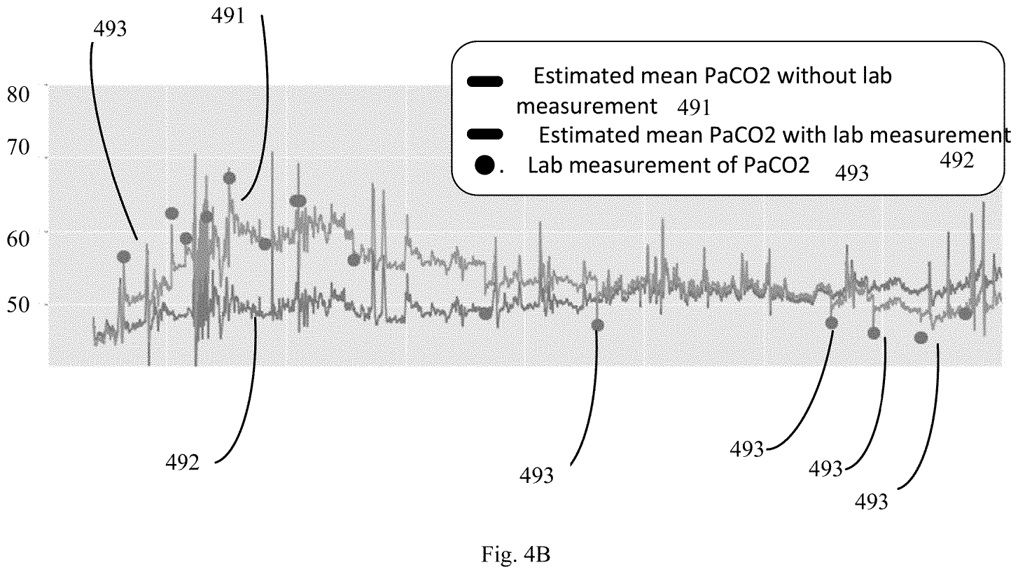

FIG. 4A depicts a non-limiting example of the physiology observer described above tracking DO2, but over a longer time interval, i.e., four (4) time steps. In the observer, the main hidden ISV is the oxygen delivery variable (DO2). The two types of measurements, Hemoglobin (Hg) and oximetry (SpO2) are in dashed circles in FIG. 4A. SpO2 is an example of the continuous or periodic measurements that the physiology observer module 122 receives from sensors, such as bedside monitors 102 and treatment devices 104 connected to the patient 101 that continuously report information. Hemoglobin (Hg) is an example of an intermittent or aperiodic measurement extracted from patient lab work that is available to the observer on a sporadic and irregular basis, and latent at times, relative to current system time. The physiology observer module 122 is capable of handling both types of measurements because, along with tracking the hidden ISVs, e.g. DO2, module 122 also continuously maintains estimates of the observed values for all types of measurements, even when measurements are not present. FIG. 4A depicts these estimates for the case of SpO2 and Hg. As can be seen, the SpO2 measurements are available regularly at each time step, whereas Hg is only available at two of the time steps.

FIG. 4B illustrates a method applying intermittent laboratory data through the physiology observer module to achieve better accuracy in an estimated ISV PDF. The specific example shows what the estimated mean of the PDF of the PaCO2 ISV is without incorporating arterial blood gases that directly measure this internal state variable, and how this estimate changes as arterial blood gas measurements are introduced into the system. Specifically, the estimated mean of the PaCO2 ISV is much closer to the actual measured PaCO2 when these measurements are incorporated as inputs.

This is achieved as follows:
The physiology observer module includes a hidden ISV called alveolar dead space which takes into account that lung ventilation may not be efficiently removing CO2 from the blood. I.e. the higher the alveolar dead space is, the higher the difference is between expired CO2 as measured by end-tidal CO2 measurement (EtCO2) and arterial CO2 as measured by PaCO2 arterial blood gases.
The PDF of this ISV is used to predict various measurements acquired from the patient, some of which might include minute ventilation, end-tidal CO2, and PaCO2 arterial blood gases
As a non-limiting example, if minute ventilation and end-tidal CO2 are continuously acquired measurements at every time tₖ₊₁, the inference engine can use Bayes theorem to update the PDF of the various ISVs, one of which is PaCO2. That is, in the formula,

$$P ( ISVs ( {t}_{k + 1} ) \vert M ( {t}_{k + 1} ) ) = \frac{P ( \begin{matrix}{m}_{1} ( {t}_{k + 1} ) , {m}_{2} ( {t}_{k + 1} ) \vert \\ ISVs ( {t}_{k + 1} )\end{matrix} ) P ( ISVs ( {t}_{k + 1} ) \vert M ( {t}_{k} ) )}{P ( {m}_{1} ( {t}_{k + 1} ) , {m}_{2} ( {t}_{k + 1} ) \vert M ( {t}_{k} ) )}$$

m₁ and, m₂ are measured values of EtCO2 and minute ventilation.
When a PaCO2 blood gas measurement is acquired at next time tₖ₊₂ in conjunction with measurements of end-tidal EtCO2 and minute ventilation, the measurement vector is augmented with the PaCO2 measurement and the above formula becomes:

$$P ( ISVs ( {t}_{k + 2} ) \vert M ( {t}_{k + 2} ) ) = \frac{P ( \begin{matrix}\begin{matrix}{m}_{1} ( {t}_{k + 2} ) , {m}_{2} ( {t}_{k + 2} ) , \\ {m}_{3} ( {t}_{k + 2} ) \vert\end{matrix} \\ ISVs ( {t}_{k + 2} )\end{matrix} ) P ( ISVs ( {t}_{k + 2} ) \vert M ( {t}_{k + 1} ) )}{P ( {m}_{1} ( {t}_{k + 2} ) , {m}_{2} ( {t}_{k + 2} ) , {m}_{3} ( {t}_{k + 2} ) \vert M ( {t}_{k + 1} ) )}$$

Where again m₁ and, m₂ are measured values of EtCO2 and minute ventilation, and m₃ is the PaCO2 measurement.
This additional information is utilized in two ways. First, the uncertainty in the PaCO2 ISV PDF is reduced (See FIG. 4B) given the more accurate direct observation of PaCO2 provided by the arterial blood gas. Second, since the PaCO2 and EtCO2 measurements are observed simultaneously, the combined information is used by the physiology observer to estimate alveolar dead-space more precisely.
When the new estimated PDF of alveolar dead-space is propagated forward in time by the dynamic model, the accuracy of the estimated PaCO2 PDF is improved even when m₃, the PaCO2 measurement, is not present.

Measurement Contribution

FIG. 8C and FIG. 8D schematically illustrate an embodiment of a clinical trajectory interpreter module 123 using posterior probabilities (i.e., joint Probability Density Functions of the ISVs) 250, produced by the physiology observer module 122, to determine the relative contribution of the measurement of at least one internal state variable to a risk determination. More specifically, the physiology observer module 122 computes a risk (a “reference risk”) that the patient is in a specific patient state at time step tₖ₊₁ using available measurements of internal state variables from that time step, and then computes alternate risks that the patient is in a specific patient state at time step tₖ₊₁ using, respectively, revised measurements of internal state variables from that time step.

FIG. 8C schematically illustrates an embodiment of a clinical trajectory interpreter module 123 having a measure contribution module 860, and FIG. 8D schematically illustrates embodiments of the operation of the measure contribution module 860. For purposes of illustration, operation of such a clinical trajectory interpreter module 123 is described below at time step tₖ₊₁, which time step tₖ₊₁ follows one or more preceding time steps, e.g., tₖ, tₖ₋₁, tₖ₋₂, tₖ₋₃, etc.

The measure contribution module 860 identifies the measurements in a patient's history that are causing the current (at time step tₖ₊₁) risk calculations or indexes, to be elevated. In the Bayesian framework utilized by the presented technology, each measurement and its history can be viewed as independent evidence contributing to the probability of a patient state. The contribution module 860 evaluates the importance, or weight or influence of one or more measurements on the patient state(s). Generally stated: this is accomplished by reversing the recursive Bayesian update process, by which evidence is incorporated into the risk calculation, and assessing the change in risk after the removal of a set of one or more measurements (each of which measurements may be referred to as a “measurement of interest”), or replacement of each measurement of interest with a different value, and its history. In some embodiments, the set of one or more measurements is a set of current measurements (e.g., measurements of a set of internal state variable at time step tₖ₊₁), and in other embodiments the set of one or more measurements is a set or sequence of measurements (e.g., of a specific internal state variable) over a particular, specified time period in the past.

Specifically, as schematically illustrated in FIG. 8D, the measure contribution module 860 takes as an input 861 the probability densities of the internal state variables 250 produced by the physiology observer module 122 for time step tₖ₊₁ (“first posterior probabilities”), which have been created using all of the measurement data available up to that time step tₖ₊₁ (which time step, in some embodiments, may be referred-to as “current time”). Based on the first posterior probabilities, the system 100 calculates a first set of clinical risks 867 (the “previously-calculated clinical risks”) for the patient 101, pursuant to processes and methods described above for generating clinical risks.

For each measurement of interest, the method implemented by the measure contribution module 860 alters one or more of the probability densities of the internal state variables 250 at 862 by either (i) removing 863 the impact of that measurement of interest on the density or (ii) substituting 864 (i.e., removing and replacing) the original value of the measurement of interest with a measurement (“substitute measurement”) equal to the nominal value of the underlying internal state variable.

Measurement Removal and Computation of Alternative Clinical Risk (Step 863 and 865)

Some embodiments calculate an alternative clinical risk of the patient being in a specific patient state by removing one measurement used in generation of a previous clinical risk, or substituting a null value for one measurement used in generation of a previous clinical risk, and computing the alternative clinical risk using the rest of the datums used in the generation of that previous clinical risk.

Measurement Substitution and Computation of Alternative Clinical Risk (Step 864 and 865)

Some embodiments calculate an alternative clinical risk of the patient being in a specific patient state by replacing one measurement used in generation of a previous clinical risk with a nominal value of said measurement, and computing the alternative clinical risk using the rest of the datums used in the generation of that previous clinical risk.

After altering one or more of the probability densities of the internal state variables 250 at 862, the method calculates revised probability densities of the internal state variables 250 (“second posterior probabilities”). Consequently, either option 863, 864 results in a new density estimate.

With this new density estimate, the calculation then calculates 865 a set of alternative risks (“alternative clinical risks”) for the patient 101, pursuant to processes and methods described above for generating clinical risks.

Next, the measure contribution module 860 compares 866 these alternative clinical risks with the previously-calculated risks 867 (which may be referred-to as “reference risks” or “actual” risks or “current risks”) that were computed for time step tₖ₊₁. The difference between the alternative clinical risks and previously-calculated clinical risks 867 is a quantitative measure of the importance of the set of measurements of interest.

At 868, the measure contribution module 860 generates another alternative risk calculation by repeating 862 and 865, and generates another risk comparison at 866.

Once the quantitative measure of the importance is determined, the rank or order of measurement importance is determined 868 by sorting the importance values from largest to smallest. This importance value and rank is sent to the display and notifications 124 system module.

The removal 863 of the impact of a measurement of interest on a corresponding probability density function, and ultimately on clinical risk(s), can be accomplished in a variety of ways.

One method is a manual re-processing of the measurement history and re-calculation of the density with a measurement of interest removed from the sequence of data.

Another method, which is the approach utilized by some illustrative embodiments, is an analytical calculation that removes the impact of a measurement (a “measurement of interest”) from the density estimate. The measurement of interest to be removed can be from the most recent set of data collected on a patient, or it may have been collected at some point in the patient's past history, e.g., a laboratory blood sample that was collected several hours ago. Some embodiments, also allow for a series of a particular measurements of interest data to be removed, e.g. 30 minutes of continuous SpO2 measurements.

The analytical methodology of operation 863 is as follows. First, based on the conditional independence employed by the underlying model, the joint distribution of the current ISVs at tₖ₊₁ and the ISVs at some in the past, t_{k+1-L}, conditioned on all of the data up to the current time M(tₖ₊₁), is given by:

P(ISVs(tₖ₊₁),ISVs(t_{k+1-L})|M(tₖ₊₁))=P(ISVs(tₖ₊₁))|ISVs(t_{k+1-L}))P(ISVs(t_{k+1-L})|M(tₖ₊₁))

Similarly, defining M(tₖ₊₁)\m(t_{k+1-L}) as the sequence of measurements M(tₖ₊₁) up to the current time with the measurement of interest, m(t_{k+1-L}), removed, the joint distribution of the current ISVs at tₖ₊₁ and the ISVs at some in the past, t_{k+1-L} conditioned on M(tₖ₊₁)\m(t_{k+1-L}), can written as:

P(ISVs(tₖ₊₁),ISVs(t_{k+1-L})|M(tₖ₊₁)\m(t_{k+1-L}))+P(ISVs(tₖ₊₁))|ISVs(t_{k+1-L}))P(ISVs(t_{k+1-L})|M(tₖ₊₁)\m(t_{k+1-L}))

Now, utilizing the conditional independence of the underlying model and Bayes' Rule, P(ISVs(tₖ₊₁), ISVs(t_{k+1-L})|M(tₖ₊₁)) can be re-written as:

$$P ( ISVs ( {t}_{k + 1} ) , ISVs ( {t}_{k + 1 - L} ) \vert M ( {t}_{k + 1} ) ) = \\ P ( ISVs ( {t}_{k + 1} ) ) \vert ISVs ( {t}_{k + 1 - L} ) ) \frac{P ( \begin{matrix}m ( {t}_{k + 1 - L} ) \vert \\ ISVs ( {t}_{k + 1 - L} )\end{matrix} ) P ( \begin{matrix}ISVs ( {t}_{k + 1 - L} ) \vert \\ M ( {t}_{k + 1} ) \text{∖} m ( {t}_{k + 1 - L} )\end{matrix} )}{P ( m ( {t}_{k + 1 - L} ) \vert M ( {t}_{k + 1} ) \text{∖} m ( {t}_{k + 1 - L} ) )}$$

From this expression and the prior expression, it can be seen that removing the influence of a measurement of interest from the joint distribution can be accomplished by dividing by the likelihood of the measurement and multiplying by the normalization, as shown below:

$$P ( ISVs ( {t}_{k + 1} ) , ISVs ( {t}_{k + 1 - L} ) \vert M ( {t}_{k + 1} ) ) \text{∖} m ( {t}_{k + 1 - L} ) ) = \frac{P ( \begin{matrix}ISVs ( {t}_{k + 1} ) , ISVs ( {t}_{k + 1 - L} ) \vert \\ M ( {t}_{k + 1} )\end{matrix} )}{P ( m ( {t}_{k + 1 - L} ) \vert ISVs ( {t}_{k + 1 - L} ) )} P ( m ( {t}_{k + 1 - L} ) \vert M ( {t}_{k + 1} ) \text{∖} m ( {t}_{k + 1 - L} ) )$$

From this joint distribution, the re-calculated density without the influence of the measurement can be recovered via marginalization,

P(ISVs(tₖ₊₁)|M(tₖ₊₁)\m(t_{k+1-L}))=∫_{ISVs∈ISV}P(ISVs(tₖ₊₁),ISVs(t_{k+1-L})|M(tₖ₊₁)\m(t_{k+1-L}))dISVs

Note in the case where L=0, i.e. the measurement of interest was taken at the current calculation time, the calculation above simplifies to:

$$P ( ISVs ( {t}_{k + 1} ) \vert M ( {t}_{k + 1} ) \text{∖} m ( {t}_{k + 1 - L} ) ) = \frac{P ( \begin{matrix}ISVs ( {t}_{k + 1} ) \vert \\ M ( {t}_{k + 1} )\end{matrix} )}{P ( \begin{matrix}m ( {t}_{k + 1} ) \vert \\ ISVs ( {t}_{k + 1} )\end{matrix} )} P ( m ( {t}_{k + 1} ) \vert M ( {t}_{k + 1} ) \text{∖} m ( {t}_{k + 1 - L} ) )$$

Because of integrals involved, the calculation outlined above is only able to be calculated exactly for certain classes of densities, for example Gaussian densities over continuous variables, and densities where the underlying variables are all discrete. In the presented technology, because Gaussian densities are used, in conjunction with the Kalman Filter (Extended version), the above calculation is tractable.

First, the joint density of the current ISVs at tₖ₊₁ and the ISVs at some time in the past, t_{k+1-L}, is formed via a method called Delay State Augmentation, in which a copy of the state information that represents the past internal state variables is augmented to the set of internal state variables at the current time, i.e. ISVs*(tₖ₊₁)={ISVs(tₖ₊₁), ISVs(t_{k+1-L})}. The probability density that is calculated by the Physiology observer module 122, is now expanded to represent both the density of the current ISVs and the density of the past augmented ISVs. The Kalman Filter used by the Physiology Observer module 122 represents the ISVs probability density as P(ISVs(tₖ₊₁)|M(tₖ₊₁))=Normal ( [symbol] ₜ_{ₖ₊₁}_{|t}_{ₖ₊₁},Σₜ_{ₖ₊₁}_{|t}_{ₖ₊₁}), where [symbol] ₜ_{ₖ₊₁}_{|t}_{ₖ₊₁} represents the state estimate at the current time (tₖ₊₁) conditioned on all the measurements up to that time, and Σₜ_{ₖ₊₁}_{|t}_{ₖ₊₁} is the corresponding covariance matrix. The density expansion is accomplished by concatenating the current state estimate with the past state estimate, and concatenating the corresponding current and past state Covariance Matrices, i.e.

$${{t}_{k + 1} \vert {t}_{k + 1}}_{*}^{} = \{ {{t}_{k + 1} \vert {t}_{k + 1} ,}_{} {{t}_{k + 1} - L \vert {t}_{k + 1}}_{} \} , {Σ}_{{t}_{k + 1} \vert {t}_{k + 1}}^{*} = [ \begin{matrix}{\sum }_{{t}_{k + 1} \vert {t}_{k + 1}} & {\sum }_{{t}_{k + 1} {t}_{k + 1 - L} \vert {t}_{k + 1}} \\ {\sum }_{{t}_{k + 1 - L} {t}_{k + 1} \vert {t}_{k + 1}} & {\sum }_{{t}_{k + 1 - L} \vert {t}_{k + 1}}\end{matrix} ] .$$

Note at the time of concatenation, the current state equals the past state, and so the state estimates are perfectly correlated. Also note, the density of these augmented internal state variables is not propagated over time as they are static, however it is updated with new measurements as they are incorporated into the density estimates.

After the augmentation, the measurement removal process is accomplished is by calculating the state estimate and Covariance matrix without the measurement of interest, m(t_{k+1-L}), or [symbol] ₜ_{ₖ₊₁}_{|{t}_{ₖ₊₁}_{\t}_{_{k+1-L}}_{}},Σₜ_{ₖ₊₁}_{|{t}_{ₖ₊₁}_{\t}_{_{k+1-L}}_{}} via the following equations:

$${{t}_{k + 1} \vert \{ {t}_{k + 1} \text{∖} {t}_{k + 1 - L} \}}_{*}^{} = {( {Σ}_{{t}_{k + 1} \vert {t}_{k + 1}}^{* - 1} - {H}^{{*}^{T}} {R}^{- 1} {H}^{*} )}^{- 1} ( {Σ}_{{t}_{k + 1} \vert {t}_{k + 1}}^{* - 1} {{t}_{k + 1} \vert {t}_{k + 1}}_{*}^{} - {H}^{{*}^{T}} {R}^{- 1} m ( {t}_{k + 1 - L} ) )$$

$${Σ}_{{t}_{k + 1} \vert \{ {t}_{k + 1} ∖ {t}_{k + 1 - L} \}}^{* - 1} = {Σ}_{{t}_{k + 1} \vert {t}_{k + 1}}^{* - 1} - {H}^{{*}^{T}} {R}^{- 1} {H}^{*}$$

Note, these equations can be derived from the Information form of the Kalman Filter update equations, presented in detail in (Jazwinski, p. 197). Having calculated these quantities, the Measure contribution calculation can now calculate the alternative Risk for comparison with the current Risk to understand the importance of the measurement of interest, measurement of interest, m(t_{k+1-L}), on the current risk level.

In the case of measurement substitution 864, it is a matter of removing the influence of the original measurement value and reapplying the new nominal value to the estimate by manipulating the equations presented for [symbol] ₜ_{ₖ₊₁}_{|{t}_{ₖ₊₁}_{\t}_{_{k+1-L}}_{}},Σₜ_{ₖ₊₁}_{|{t}_{ₖ₊₁}_{\t}_{_{k+1-L}}_{}}.

FIG. 8E is a flowchart of an embodiment of a method 880 of assessing measurement contribution to the determination that a patient is in a specified patient sate.

Step 881 includes generating, at the current time step, first joint probability functions (or first posterior probabilities) 250 of a set of internal state variables for a specific patient, as described above in connection with the Physiology Observer Module 122. The first joint probability functions (or first posterior probabilities) 250 of a set of internal state variables generally includes a plurality of first joint probability functions, each joint probability function being a probability density function corresponding to an internal state variable obtained from the patient, for example, by a Data Reception Module 121.

### Example: Inadequate Oxygen Delivery

For example, for assessing the patient state of inadequate oxygen delivery in a patient, for which the corresponding hidden ISV is mixed venous oxygen saturation, the Data Reception Module 121 obtains, from the patient the patient's heart rate (using a heart rate sensor) and the patient's SpO2 (using a pulse oximeter), and passes that information to the Observation Model 221, and the Physiology Observer Module 122 and the Clinical Trajectory Interpreter Module 123 operate as described above.

### Example: Inadequate Ventilation of Carbon Dioxide

As another example, when assessing inadequate ventilation of carbon dioxide (IVCO2 Index) for which the corresponding hidden ISV is arterial partial pressure of carbon dioxide blood (PaCO2), the Data Reception Module 121 obtains, from the patient, the patient's heart rate (using a heart rate sensor) and the patient's SpO2 (using a pulse oximeter), and the patient's respiratory rate (using a respiratory rate sensor), and passes that information to the Observation Model 221, and the Physiology Observer Module 122 and the Clinical Trajectory Interpreter Module 123 operate as described above.

### Example: Acidosis

As another example, when assessing the patient state of Acidosis, for which the hidden internal state variable is Arterial blood pH, the Data Reception Module 121 obtains, from the patient, heart rate, SpO2 level, and respiratory rate and passes that information to the Observation Model 221, and the Physiology Observer Module 122 and the Clinical Trajectory Interpreter Module 123 operate as described above.

### Example: Hyperlactatemia

As another example, when assessing the patient state of Hyperlactatemia (LA Index), for which the hidden internal state variable is arterial lactate level (or in some embodiments whole blood lactate level), the Data Reception Module 121 obtains, from the patient, heart rate and SpO2 level and passes that information to the Observation Model 221, and the Physiology Observer Module 122 and the Clinical Trajectory Interpreter Module 123 operate as described above.

Step 882 includes generating a clinical risk that the patient is in the specified patient state, as described above in connection with the Clinical Trajectory Interpreter Module 123, using the first joint probability functions 250 as input.

Step 883 includes generating, at the current time step, an alternative set of first joint probability functions (or first posterior probabilities) 250 of a set of internal state variables for the specific patient. Step 883 may be implemented in several different ways.

For example, in one embodiment, the method 880 changes one or more of the measurement obtained by the Data Reception Module 121, and then allows the Physiology Observer Module 122 and the Clinical Trajectory Interpreter Module 123 to operate as described above to produce an alternate clinical risk, at step 884.

For example, if the patient's heart rate as obtained by the Data Reception Module 121 and used in the operations of step 881 and 882 was 70 beats per minute, some embodiment change that heart rate data to another value (the “altered” value, such as another fixed rate (e.g., 50 bpm, 60 bpm, 80 bpm, 90 bpm, to name but a few examples. Other embodiments change the heart rate variable to another heart rate, such as the average heart rate for the present patient, or an average (or nominal) heart rate for a patient of the same age as the present patient, to name but a few examples. The Data Reception Module 121 the operates as described above, but assuming that the measured heartrate for this patient is the altered value to produce an alternate Conditional Likelihood Kernel 230, and the Observer Module 122 and the Clinical Trajectory Interpreter Module 123 operate as described above, ultimately producing alternate clinical risk, at step 884.

Some Variations

Other embodiments generate an alternate clinical risk not by (or in addition to) changing one or more of the measurements obtained by the Data Reception Module 121, but by changing one or more probability density functions used in the operation of the Physiology Observer Module 122. For example, some embodiments change (e.g., edit) one or more of the probability functions in the Conditional Likelihood Kernel 230 produced by the Observation model 221 (i.e., using the measurement data provided by the Data Reception Module 121 and the Predicted probability density functions of predicted probability density functions of internal state variables 211 produced in the predict step 210) by deleting, from the Conditional Likelihood Kernel 230, the probability density function corresponding to the measurement of interest. Other embodiments change (e.g., edit) one or more of the probability functions in the Conditional Likelihood Kernel 230 produced by the Observation model 221 by replacing, in the Conditional Likelihood Kernel 230, a probability density function corresponding to the measurement of interest with an alternate probability density function corresponding to the measurement of interest. Other embodiments change (e.g., edit) one or more of the probability functions of the predicted probability density functions of internal state variables 211 by deleting, from the predicted probability density functions of internal state variables 211, the probability density function corresponding to the measurement of interest.

Other embodiments change (e.g., edit) one or more of the probability functions in the predicted probability density functions of internal state variables 211 replacing, in the of the predicted probability density functions of internal state variables 211, a probability density function corresponding to the measurement of interest with an alternate probability density function corresponding to the measurement of interest.

Yet other embodiments generate an alternate clinical risk not by (or in addition to) changing one or more of the measurement obtained by the Data Reception Module 121, but by changing one or more probability density functions of the first joint probability functions 250 produced by the Physiology Observer Module 122. For example, some embodiments change (e.g., edit) one or more of the probability functions of first joint probability functions 250, by deleting, from the first joint probability functions 250, the probability density function corresponding to the measurement of interest. Other embodiments some embodiments change (e.g., edit) one or more of the probability functions of first joint probability functions 250 by replacing, in the first joint probability functions 250, a probability density function corresponding to the measurement of interest with an alternate probability density function corresponding to the measurement of interest.

Alternate Clinical Risks

Some embodiments generate one or more additional alternate clinical risks at repeating (step 885) step 883 and 884, each time changing a different measurement of interest.

For example, when the patient state is Inadequate Oxygen Delivery (for which the measured internal state variables are heart rate and SpO2), some embodiments generate a first alternate patient state by replacing (relative to a patient state generated using measured heart rate and measured SpO2) the measured heart rate measurement taken from the patient with other heart rate data (e.g., 50 bpm, 60 bpm, 70 bpm, 80 bpm, 90 bpm, to name but a few examples) and generating the patient state using that replacement heart rate data and the measured SpO2 data. Some embodiments generate a second alternate patient state by replacing (relative to a patient state generated using measured heart rate and measured SpO2) the measured SpO2 measurement taken from the patient with other SpO2 data (e.g., a null value or a nominal value, to name but a few examples) and generating the patient state using that replacement SpO2 data and the measured heart rate data.

As another example, when the patient state is Inadequate ventilation of carbon dioxide (IVCO2 Index) some embodiments generate a first alternate patient state by replacing (relative to a patient state generated using measured heart rate and measured SpO2) the measured heart rate measurement taken from the patient with other heart rate data (e.g., 50 bpm, 60 bpm, 70 bpm, 80 bpm, 90 bpm, to name but a few examples) and generating the patient state using that replacement heart rate data and the measured SpO2 data and the measured respiratory rate data. Some embodiments generate a second alternate patient state by replacing (relative to a patient state generated using measured heart rate and measured SpO2) the measured SpO2 data taken from the patient with other SpO2 data (e.g., a null value or a nominal value, to name but a few examples), and generating the patient state using that replacement SpO2 data and the measured heart rate data and the measured respiratory rate. Some embodiments generate a third alternate patient state by replacing (relative to a patient state generated using measured heart rate and measured SpO2) the measured respiratory rate data measurement taken from the patient with other respiratory rate data (e.g., a null value or a nominal value, to name but a few examples) and generating the patient state using that replacement respiratory rate data and the measured heart rate data and the measured SpO2 data.

Step 886 compares the different clinical risks from among the clinical risks generated as described above, to assess the impact of the measurements of interest on the clinical risk. For example, in some embodiments, step 886 compares (a) a clinical risk generated using data obtained by the Data Reception Module 121 without alternation (which may be referred-to as a “reference” clinical risk), to (b) an alternate clinical risk corresponding to a measurement of interest generated at steps by steps 883 and 884. For example, such a comparison may subtract one such clinical risk from another to determine the quantitative difference (or “delta”) between them. Some embodiments additionally computer the absolute value of the quantitative difference between them to determine the magnitude of that difference.

Some embodiment compare more than one alternative clinical risk (e.g., each produced via step 883 and 884) against a clinical risk generated using data obtained by the Data Reception Module 121 (which may be referred-to as a “reference” clinical risk) without alternation. Step 886 can then order the quantitative differences resulting from such comparisons based, for example, on the magnitude of such differences. The difference with the smallest magnitude indicates that the measurement of interest used to produce the corresponding alternative risk is the measurement that has the least effect or impact on the patient state. The difference with the largest magnitude indicates that the measurement of interest used to produce the corresponding alternative risk is the measurement that has the greatest effect or impact on the patient state.

Step 887 then displays the results, to inform a user not only the computed patient state (generated at step 882), but also which internal state variable has the greatest effect or impact on the patient state, and or which internal state variable has the least effect or impact on the patient state. Some embodiments show the respective effect (or impact) of each of a plurality of internal state variables on the patient state, in order of rank from most to least impact, or least to greatest impact. For example, FIG. 8F schematically illustrates the patient's patient state 871, and the impact on that patient state of each of a plurality of variables: the graph of heart rate (874), SpO2 (875), and three values of mmHg (876, 877 and 878), which graphs are displayed in the order, from top to bottom, of the impact that each such variable has on the patient state. Some embodiments also include a drop-down menu 879 listing those internal state variables.

### ILLUSTRATIVE EXAMPLES

The following examples illustrate the operations of the system described above.

Illustrative Example 1 (measurements of two ISVs). A method of transforming measured data of a patient into data for a particular patient state based on a generated internal state variable includes:

providing a plurality of sensors including at least a first sensor and a second sensor, to measure a corresponding plurality of internal state variables V_{B}, B=1, 2 . . . C, the plurality of sensors physically attached to the patient;

substantially continuously acquiring, by a computer over a series of time steps t_{K}, K=0, 1, . . . Z, from the plurality of sensors connected with the patient, a set of as-measured datums m_{S}, S=1, 2 of internal state variables, including a first as-measured datum (m₁) for a first internal state variable (V₁) at time step tₖ₊₁, and a second as-measured datum (m₂) for a second internal state variable (V₂) at time step tₖ₊₁;

generating a reference risk by the computer using the set of as-measured datums (m₁, m₂) from time step tₖ₊₁, a reference conditional likelihood kernel for the internal state variables V_{B} at time tₖ₊₁, the reference conditional likelihood kernel comprising a set of probability density functions of the internal state variables V_{B} for the time step tₖ₊₁, each of the internal state variables describing a parameter physiologically relevant to the particular patient state of said patient at time step tₖ₊₁;

generating, with the computer and using Bayes theorem, reference posterior predicted conditional probability density functions for the plurality of the internal state variables V_{B} for the time step tₖ₊₁ given the reference conditional likelihood kernel for the internal state variables V_{B} at time tₖ₊₁ and predicted probability density functions of each of the internal state variables V_{B} predicted from a preceding time step tₖ for time step tₖ₊₁; and

generating, from the reference posterior predicted conditional probability density functions, a reference function of the generated internal state variable;

identifying, with the computer, from the reference function of the generated internal state variable, a reference risk that the patient is in the particular patient state;

and generating a first alternate risk by:

editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A});

generating, by the computer using the first alternate set of datums, a first alternate conditional likelihood kernel for the internal state variables V_{B} at time tₖ₊₁, the first alternate conditional likelihood kernel comprising a first alternate set of probability density functions of the internal state variables V_{B} predicted from a preceding time step tₖ for the time step tₖ₊₁;

generating, with the computer and using Bayes theorem, first alternate posterior predicted conditional probability density functions for the plurality of the internal state variables V_{B} for the time step tₖ₊₁ given the first alternate conditional likelihood kernel for the internal state variables V_{B} at time tₖ₊₁ and the predicted probability density functions of each of the internal state variables V_{B} for time step tₖ₊₁;

generating, from the first alternate posterior predicted conditional probability density functions, a first alternate function of the generated internal state variable; and

identifying, with the computer, from the first alternate function of the generated internal state variable, a first alternate risk that the patient is in the particular patient state at time step tₖ₊₁, said first alternate risk associated with said first internal state variable V₁;

and generating a second alternate risk by:

editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A});

generating, by the computer using the second alternate set of datums, a second alternate conditional likelihood kernel for the internal state variables V_{B} at time tₖ₊₁, the second alternate conditional likelihood kernel comprising a second alternate set of probability density functions of the internal state variables V_{B} predicted from a preceding time step tₖ for the time step tₖ₊₁;

generating, with the computer and using Bayes theorem, second alternate posterior predicted conditional probability density functions for the plurality of the internal state variables V_{B} for the time step tₖ₊₁ given the second alternate conditional likelihood kernel for the internal state variables V_{B} at time tₖ₊₁ and the predicted probability density functions of each of the internal state variables V_{B} for time step tₖ₊₁; and

generating, from the second posterior predicted conditional probability density functions, a second alternate function of the generated internal state variable; and

identifying, with the computer, from the second alternate function of the generated internal state variable, a second alternate risk that the patient is in the particular patient state at time step tₖ₊₁, said second alternate risk associated with said second internal state variable (V₂);

determining, as among the as-measured datums, which as-measured datum has the quantitatively greatest influence on the reference risk (that the patient is in the particular patient state at time step tₖ₊₁) by:

comparing the first alternate risk that the patient is in the particular patient state to the reference risk to produce a first delta associated with the first internal state variable, and by

comparing the second alternate risk that the patient is in the particular patient state to the reference risk to produce a second delta associated with the second internal state variable,

the as-measured datum having the quantitatively greatest influence on the reference risk being the as-measured datum associated with the larger of the first delta and the second delta; and

displaying, on a graphical user interface, the reference risk that the patient is in the particular patient state at time step tₖ₊₁, and an identifier of the as-measured datum has the quantitatively greatest influence on the reference risk at time step tₖ₊₁.

Illustrative Example 2 (measurements of three ISVs): A method of transforming measured data of a patient into data for a particular patient state based on a generated internal state variable includes:

providing a plurality of sensors including at least a first sensor and a second sensor and a third sensor, to measure a corresponding plurality of internal state variables V_{B}, B=1, 2, 3 . . . C, the plurality of sensors physically attached to the patient;

substantially continuously acquiring, by a computer over a series of time steps t_{K}, K=0, 1, . . . Z, from the plurality of sensors connected with the patient, a set of as-measured datums m_{S}, S=1, 2, 3 of internal state variables, including a first as-measured datum (m₁) for a first internal state variable (V₁) at time step tₖ₊₁, and a second as-measured datum (m₂) for a second internal state variable (V₂) at time step tₖ₊₁, and a third as-measured datum (m₃) for a third internal state variable (V₃) at time step tₖ₊₁;

generating a reference risk by the computer using the set of as-measured datums (m₁, m₂, m₃) from time step tₖ₊₁, a reference conditional likelihood kernel for the internal state variables V_{B} at time tₖ₊₁, the reference conditional likelihood kernel comprising a set of probability density functions of the internal state variables V_{B} for the time step tₖ₊₁, each of the internal state variables describing a parameter physiologically relevant to the particular patient state of said patient at time step tₖ₊₁;

generating, with the computer and using Bayes theorem, reference posterior predicted conditional probability density functions for the plurality of the internal state variables V_{B} for the time step tₖ₊₁ given the reference conditional likelihood kernel for the internal state variables V_{B} at time tₖ₊₁ and predicted probability density functions of each of the internal state variables V_{B} predicted from a preceding time step tₖ for time step tₖ₊₁; and

generating, from the reference posterior predicted conditional probability density functions, a reference function of the generated internal state variable;

identifying, with the computer, from the reference function of the generated internal state variable, a reference risk that the patient is in the particular patient state;

and generating a first alternate risk by:

editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A});

generating, by the computer using the first alternate set of datums, a first alternate conditional likelihood kernel for the internal state variables V_{B} at time tₖ₊₁, the first alternate conditional likelihood kernel comprising a first alternate set of probability density functions of the internal state variables V_{B} predicted from a preceding time step tₖ for the time step tₖ₊₁;

generating, with the computer and using Bayes theorem, first alternate posterior predicted conditional probability density functions for the plurality of the internal state variables V_{B} for the time step tₖ₊₁ given the first alternate conditional likelihood kernel for the internal state variables V_{B} at time tₖ₊₁ and the predicted probability density functions of each of the internal state variables V_{B} for time step tₖ₊₁;

generating, from the first alternate posterior predicted conditional probability density functions, a first alternate function of the generated internal state variable; and

identifying, with the computer, from the first alternate function of the generated internal state variable, a first alternate risk that the patient is in the particular patient state at time step tₖ₊₁, said first alternate risk associated with said first internal state variable V₁;

and generating a second alternate risk by:

editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A});

generating, by the computer using the second alternate set of datums, a second alternate conditional likelihood kernel for the internal state variables V_{B} at time tₖ₊₁, the second alternate conditional likelihood kernel comprising a second alternate set of probability density functions of the internal state variables V_{B} predicted from a preceding time step tₖ for the time step tₖ₊₁;

generating, with the computer and using Bayes theorem, second alternate posterior predicted conditional probability density functions for the plurality of the internal state variables V_{B} for the time step tₖ₊₁ given the second alternate conditional likelihood kernel for the internal state variables V_{B} at time tₖ₊₁ and the predicted probability density functions of each of the internal state variables V_{B} for time step tₖ₊₁; and

generating, from the second posterior predicted conditional probability density functions, a second alternate function of the generated internal state variable; and

identifying, with the computer, from the second alternate function of the generated internal state variable, a second alternate risk that the patient is in the particular patient state at time step tₖ₊₁, said second alternate risk associated with said second internal state variable (V₂);

and generating a third alternate risk by:

editing the set of as-measured datums by replacing the third as-measured datum (m₃) with a third alternate datum value to produce a third alternate datum (m_{3A}), the third alternate datum value distinct from the as-measured value for the third as-measured datum (m₂), to produce a third alternate set of datums including the first as-measured datum (m₁), the third as-measured datum (m₃) and the third alternate datum (m_{3A});

generating, by the computer using the third alternate set of datums, a third alternate conditional likelihood kernel for the internal state variables V_{B} at time tₖ₊₁, the third alternate conditional likelihood kernel comprising a third alternate set of probability density functions of the internal state variables V_{B} predicted from a preceding time step tₖ for the time step tₖ₊₁;

generating, with the computer and using Bayes theorem, third alternate posterior predicted conditional probability density functions for the plurality of the internal state variables V_{B} for the time step tₖ₊₁ given the third alternate conditional likelihood kernel for the internal state variables V_{B} at time tₖ₊₁ and the predicted probability density functions of each of the internal state variables V_{B} for time step tₖ₊₁; and

generating, from the third posterior predicted conditional probability density functions, a third alternate function of the generated internal state variable; and

identifying, with the computer, from the third alternate function of the generated internal state variable, a third alternate risk that the patient is in the particular patient state at time step tₖ₊₁, said third alternate risk associated with said third internal state variable (V₃);

determining, as among the as-measured datums, which as-measured datum has the quantitatively greatest influence on the reference risk (that the patient is in the particular patient state at time step tₖ₊₁) by:

comparing the first alternate risk that the patient is in the particular patient state to the reference risk to produce a first delta associated with the first internal state variable, and by

comparing the second alternate risk that the patient is in the particular patient state to the reference risk to produce a second delta associated with the second internal state variable,

comparing the third alternate risk that the patient is in the particular patient state to the reference risk to produce a third delta associated with the second internal state variable,

the as-measured datum having the quantitatively greatest influence on the reference risk being the as-measured datum associated with the larger of the first delta and the second delta and the third delta; and

displaying, on a graphical user interface, the reference risk that the patient is in the particular patient state at time step tₖ₊₁, and an identifier of the as-measured datum has the quantitatively greatest influence on the reference risk at time step tₖ₊₁.

In illustrative embodiments, an as-measured datum is associated with a delta when the delta is produced by comparing (i) the reference risk to (ii) the alternate risk produced by altering (e.g., step 862) the as-measured datum. For example, if the first delta (produced by comparing the reference risk and the first alternate risk) is the largest delta (e.g., as between the first delta and second delta, or as between the first delta, second delta, and third delta), then the as-measured datum associated with that first a delta is the first as-measured datum (m₁) [since the first as-measured datum was the altered (e.g., replaced with an alternate datum at step 862) to produce the first alternative risk and subsequently to produce the first delta]. If the second delta (produced by comparing the reference risk and the second alternate risk) is the largest delta (e.g., as between the first delta and second delta, or as between the first delta, second delta, and third delta), then the as-measured datum associated with that second delta is the second as-measured datum (m₂) [since the second as-measured datum was the altered (e.g., replaced with an alternate datum at step 862) to produce the second alternative risk and subsequently to produce the second delta]. Similarly, if the third delta (produced by comparing the reference risk and the third alternate risk) is the largest delta (e.g., as between the first delta, second delta, and third delta), then the as-measured datum associated with that third a delta is the third as-measured datum (m₃) [since the third as-measured datum was the altered (e.g., replaced with an alternate datum at step 862) to produce the third alternative risk and subsequently to produce the third delta].

FIG. 8F schematically illustrates an embodiment of a display 870 showing influence of internal state variables on a patient's clinical risk. This particular embodiment shows the impact of certain internal state variables on a patient's clinical risk of low IDO2. Specifically, this illustrative embodiment shows the impact of Heart Rate (“HR”), SpO2, SAP, MAP and DAP on the patient's risk of low IDO2, although the principles apply to any display of clinical risk and its associated internal state variable. As shown, FIG. 8F depicts one embodiment 870 of the display of the influence of an internal state variable or measurement of an internal state variable on a patient's clinical risk. The display 870 shows the clinical risk plotted as a bar graph vs time (871) at the top of the figure, as well as the monitored measurements (874, 875, 876, 877, 878) for the patient plotted vs time below the risk bar graph 871. When the user hovers over the clinical risk graph 871, the measurements or ISVs that are contributing to the clinical risk level at that time are displayed in a box 879. The box 879 in the figure displays a non-limiting example of the top 3 contributors listed in order based on the amount of influence each contributor has had on the displayed risk level.

### Example: FIG. 9

FIG. 9 illustrates a non-limiting example of a definition of a patient state that may be employed by the clinical trajectory interpreter module 123. Specifically, it assumes that the function P(S|ISV₁, ISV₂, . . . , ISVₙ) may be defined by partitioning the domain spanned by the internal state variables ISV₁, ISV₂, . . . , ISVₙ. The particular example assumes that the patient physiology is described by two internal state variables: Pulmonary Vascular Resistance (PVR) and Cardiac Output (CO). The particular risks and respective etiologies that may be captured by these two ISVs emanate from the effects of increased pulmonary vascular resistance on the circulation. Specifically, high PVR may cause right-heart failure and consequently reduced cardiac output. Therefore, PVR can be used to define the attributes of Normal PVR and High PVR, and CO to define the attributes of Normal CO and Low CO, by assigning thresholds with the two variables. By combining these attributed, four separate states can be defined: State 1: Low CO, Normal PVR; State 2: Low CO, High PVR; State 3: Normal CO, High PVR; State 4: Normal CO Normal PVR.

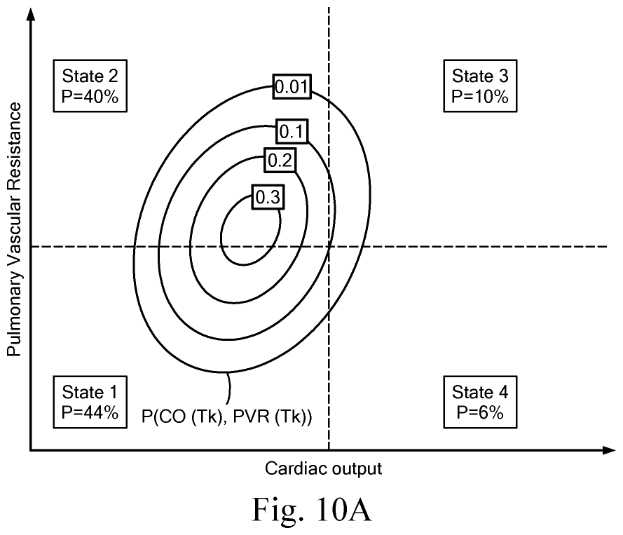

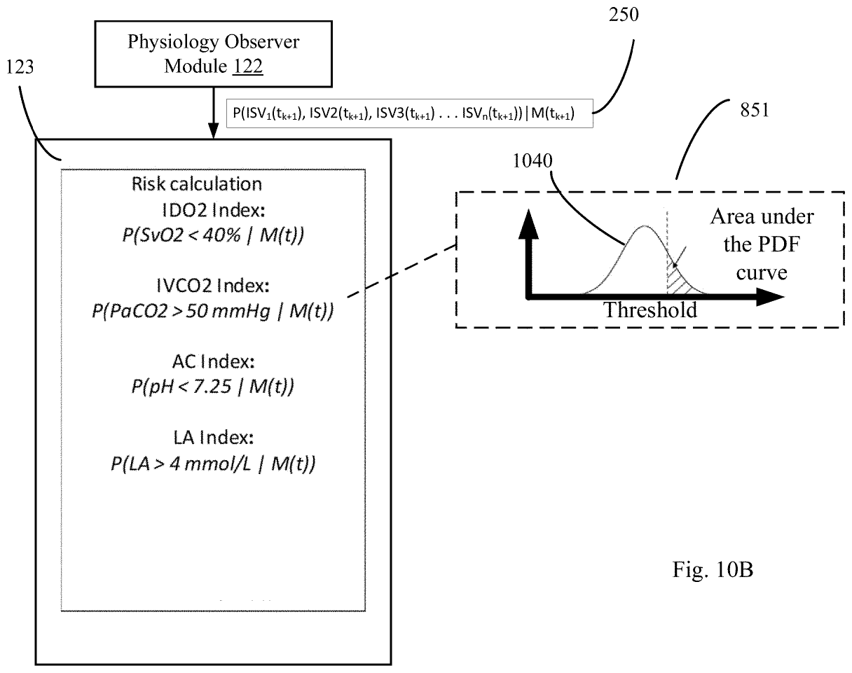

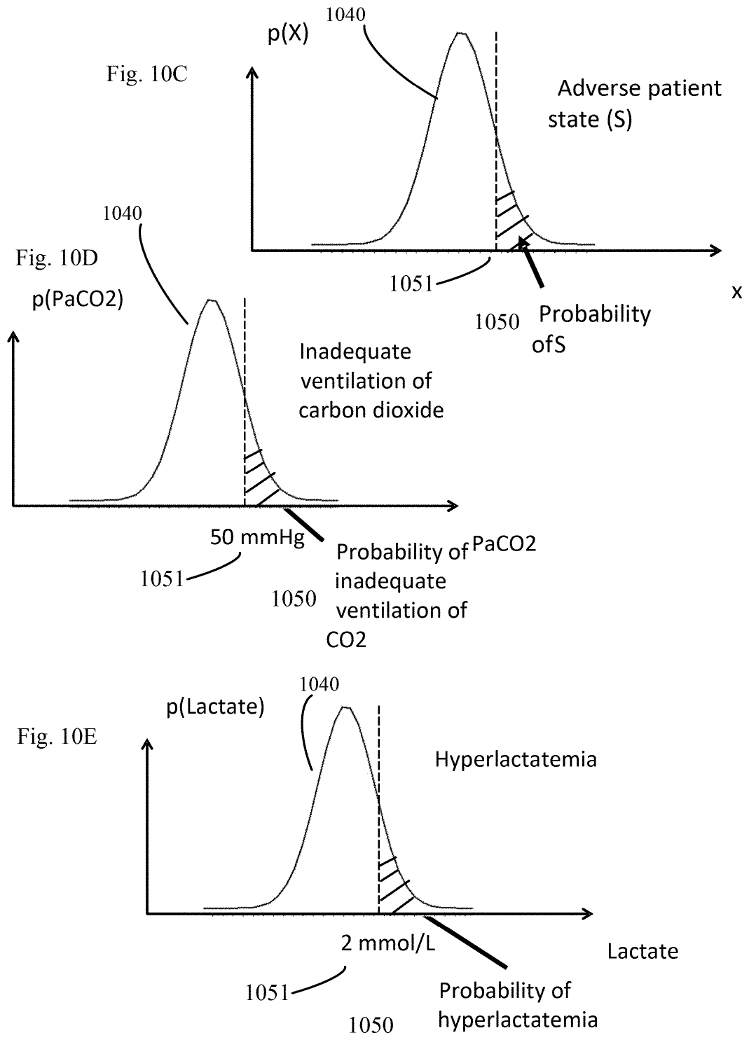

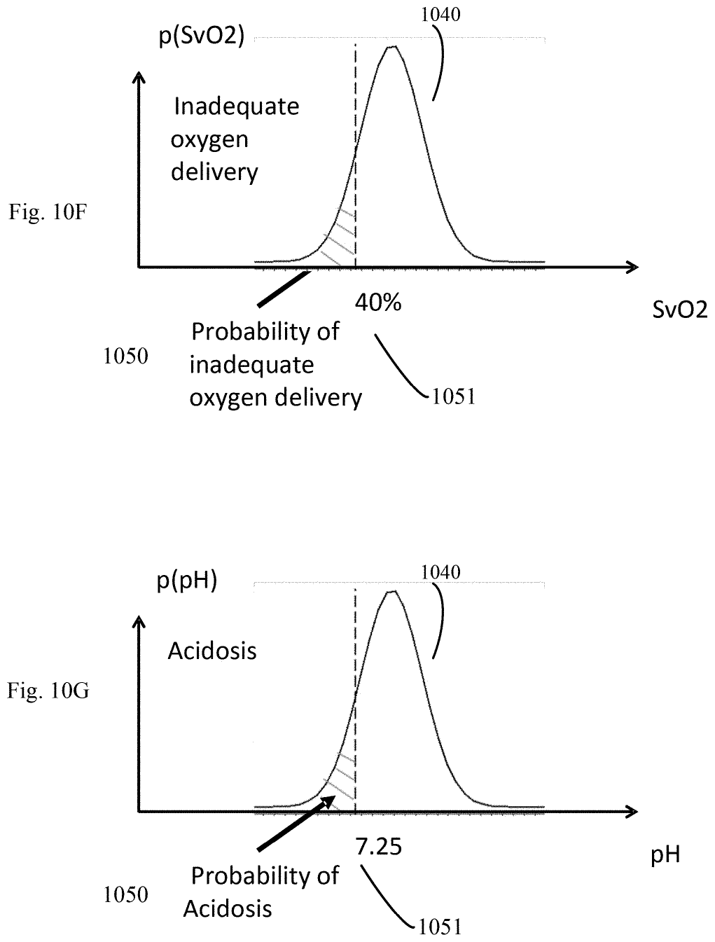

### Example: FIG. 10A

FIG. 10A illustrates a non-limiting example of how the clinical trajectory interpreter module 123 may employ the definition of patient states to assign probabilities that the patient may be classified under each of the four possible patient states at a particular point of time. In the example, the clinical trajectory interpreter module 123 takes the joint probability density function of P(Cardiac Output (Tk), Pulmonary Vascular Resistance (Tk)) and integrates it over the regions corresponding to each particular state, which produces P(S1(Tk)), P(S2(Tk)), P(S3(Tk)), and P(S4(Tk)). In this way, the clinical trajectory interpreter module 123 assigns a probability that a particular patient state is ongoing, given the information provided by the physiology observer module 122. Note that if the output of the physiology observer module 122 is not a closed form function 260 but a histogram 280 of particles 270, the clinical interpreter will not perform integration but just calculate the relative fraction of particles 270 within each region.

FIG. 10B illustrates an embodiment of a Clinical Trajectory Interpreter 123 module that may be referred-to as a Risk Calculation module. The Risk Calculation depicted calculates the probability that particular internal state variables that represent key bio-markers, e.g. SvO2 or PaCO2, are abnormal, i.e. above or below particular pre-defined clinically significant values at a particular time (e.g., in keeping with illustrative embodiments, time tₖ₊₁). This risk calculation is different from, and distinguishable from, the operation hazard level assignment 802 of FIG. 8A. Specifically, the Risk calculation takes as an input, the continuous probability densities 250 estimated by the Physiology observer module 122 for a particular time step, and calculates the cumulative probabilities of interest, each of which may be expressed as a risk. The four probabilities calculated in the embodiment of FIG. 10B are 1) the probability of inadequate oxygen delivery (or risk that the patient is in a patient state of inadequate oxygen delivery) defined as a mixed venous oxygen saturation (SvO2) below 40%, also referred to as the IDO2 Index, 2) the probability of inadequate ventilation of carbon dioxide (or risk that the patient is in a patient state of inadequate ventilation of carbon dioxide), defined as arterial partial pressure greater than 50 mmHg, also referred to as the IVCO2 Index, 3) the probability of acidosis (or risk that the patient is in a patient state of acidosis), defined as blood pH below 7.25, also referred to as the AC Index, and 4) the probability of hyperlactatemia (or risk that the patient is in a patient state of hyperlactatemia), defined as lactate blood levels greater than 4.0 mmol/L, also referred to as the LA Index.

Various patient states (adverse medical conditions), their associate internal state variables, and sensors included in a set of sensors supplying patient measurements to the system 100 are listed in Table 1, below.

| Patient State | Hidden ISV | Sensor(s) |
|---|---|---|
| Inadequate Oxygen | mixed venous | a heart rate sensor |
| Delivery | oxygen saturation | and an SpO2 sensor, |
| Inadequate ventilation of | arterial partial | a heart rate sensor |
| carbon dioxide (IVCO2 | pressure of | and an SpO2 sensor, |
| Index) defined as a patient's | carbon dioxide | respiratory rate |
| arterial partial pressure of | blood PaCO2 | sensor |
| carbon dioxide blood being | | |
| greater than a particular | | |
| value, e.g. 50 mmHg | | |
| Acidosis (AC Index) defined | Arterial blood pH | a heart rate sensor |
| as a patient's blood pH | | and an SpO2 sensor, |
| being less than a particular | | respiratory rate |
| value, e.g. 7.25 | | sensor |
| Hyperlactatemia (LA Index) | arterial lactate | a heart rate sensor |
| defined as a patient's | level {or whole | and an SpO2 sensor |
| arterial lactate level being | blood lactate | |
| greater than a particular | level} | |
| value, e.g. 4 mmol/L | | |

Inadequate Oxygen Delivery Embodiment

As a non-limiting example, the patient state of inadequate oxygen delivery may be inferred by illustrative embodiments from a heart rate and an SpO2 sensor in the following ways. The physiology observer module 122 continuously interprets ISV data based on the following understandings:
If a patient has decreasing pulse oximetry and rising heart rate, the physiology model will infer that there is a determinable probability of rising lactate.
The model by the physiology observer in this example will capture explicitly the relationship between the ISVs of arterial saturation (measured by SpO2) and heart rate (measured by the heart rate sensor) and the hidden ISV of mixed venous oxygen saturation.
As a result, at each time instance the physiology observer will update the PDF ISV of mixed venous oxygen saturation, and because of the inferred increase in heart rate and decrease in arterial saturation, the PDF will imply higher probability for lower values of the ISV SvO2.
This in turn will be interpreted by the Clinical Interpreter module 123 as a rising risk for inadequate oxygen delivery. The risk of the patient being in a patient state of inadequate oxygen delivery may be calculated by the calculation:

IDO2 Index=P(SvO2<40%|M(tₖ))=∫_{-∞}⁴⁰P(SvO2|M(tₖ))dSvO2

Note that in the foregoing formula, the threshold is 40 percent (40%), but the that illustrative embodiment does not limit all embodiments. The threshold may be determined by the clinician or system developer or operator.

Hyperlactatemia Embodiment

Similarly, another non-limiting example is how the state of Hyperlactatemia can be calculated with the same set of sensors: i.e.:
The model in the physiology observer can relate the state of inadequate oxygen delivery as reflected by the ISV of SvO2 as the probable onset of anaerobic metabolism.
The model will take into account that the more probable it is that the patient is experiencing anaerobic metabolism, the higher is the likelihood of lactate production, which will mean that with each update the PDF of the ISV of lactate will indicate higher probable values for lactate.

In turn the clinical trajectory interpreter module 123 will compute the risk that the patient is in a patient state of hyperlactatemia as:

$$LA Index = P ( Lactate < 4 \frac{mmol}{L} \vert M ( {t}_{k} ) ) = {\int }_{- ∞}^{4} P ( Lactate \vert M ( {t}_{k} ) ) dLactate$$

Note that in the foregoing formula, the threshold is 4 mmol/L, but the illustrative embodiment does not limit all embodiments. The threshold may be determined by the clinician or system developer or operator.

Inadequate Ventilation of Carbon Dioxide Embodiment

Similarly, yet another non-limiting example is using respiratory rate sensor in addition to SpO2 and heart rate sensors to determine the probability that the patient is in a state of inadequate ventilation of carbon dioxide. In this example a rising respiratory rate and heart rate while arterial saturation stays the same may be interpreted by the model in the physiology observer module 122 as a physiologic response the probable elevation of arterial carbon dioxide. This inference will be reflected by higher probable values of the ISV of PaCO2, and the risk for the patient being in a patient state of inadequate ventilation of carbon dioxide state can then be computed by:

IVCO2 Index=P(PaCO2>50 mmHg|M(tₖ))=∫₅₀^{∞}P(PaCO2|M(tₖ))dPaCO2

Note that in the foregoing formula, the threshold for arterial partial pressure is 50 mmHg, but the illustrative embodiment does not limit all embodiments. The threshold may be determined by the clinician or system developer or operator.

Acidosis Embodiment

Finally, another non-limiting example is the computation of probability that a patient is in the state of acidosis. Acidosis can be caused both by rising PaCO2 or rising lactate. An additional effect in the model of the physiology observe which captures this relationship can then infer the rising probable values of the ISVs of lactate and PaCO2 as decreasing probable values of arterial pH as captured by the PDF of this ISV. As a result, the probability of the patient being in a patient state of acidosis can be given by:

AC Index=P(pH<7.25|M(tₖ))=∫_{-∞}^{7.25}P(pH|M(tₖ))dpH

Note that in the foregoing formula, the threshold is a pH of 7.25, but the that illustrative embodiment does not limit all embodiments. The threshold may be determined by the clinician or system developer or operator.

The variables SvO2, PaCO2, pH, and Lactate are all internal state variables, or related to internal state variables, for which the Physiology observer module 122 calculates the probability density. Note, in FIG. 10B, the dashed call-out box graphically depicts an illustrative example of this calculation.

FIG. 10C schematically illustrates a generic embodiment of this calculation, for a PDF of an ISV termed “p(X).” The probability 1050 of adverse patient state (S) (e.g., an adverse medical condition) is defined as the area under the curve 1040 above (or in some embodiments, below) a threshold 1051, where the curve 1040 represents the internal state variable, which in some embodiments is a hidden internal state variable. More specific embodiments are presented in FIG. 10D, FIG. 10E, FIG. 10F and FIG. 10G, discussed below. In general, the threshold in each such embodiments may be specified by user of the system 100 (e.g., a clinician, such as a doctor, nurse, etc.), based for example on which adverse medical condition is suspected by the clinician.

It should be noted that each of the probabilities can be calculated from densities either conditioned on contemporaneous measurement data (e.g., measurements received for time step tₖ₊₁) (as shown in the equations) or not conditioned on measurement data, allowing the system to produce these quantities regardless data availability levels. The calculations can be performed via standard numerical integration techniques, or when the functional form of the underlying densities is more complicated, Monte Carlo integration techniques can be used. In the current implementation, the densities are Gaussian and so standard software packages for computing these quantities are available.

Once calculated, these risk quantities are sent to the Display and notification system module 124 for display on a display device.

### EXAMPLES

FIG. 10D: Inadequate Ventilation of Carbon Dioxide

FIG. 10D illustrates the evaluation of the state of Inadequate Ventilation of Carbon Dioxide based on the ISV of partial pressure of arterial blood CO2 (PaCO2), which ISV is represented by curve 1040. As a non-limiting example the probability 1050 of this state is computed as the cumulative distribution of p(PaCO2) greater than the 50 mmHg threshold. The resulting Clinical Risk can be displayed as an index whose instantaneous time value is given by:

IVCO2 Index=P(PaCO2>50 mmHg|M(tₖ))=∫₅₀^{∞}P(PaCO2|M(tₖ))dPaCO2

The threshold 1051 in the example of FIG. 10D has different values in different embodiments. For example, other embodiments may use a threshold of 50 mmHg or 60 mmHg, as selected or specified by a clinician.

FIG. 10E: Hyperlactatemia

FIG. 10E illustrate the evaluation of the state of Hyperlactatemia based on the ISV of whole blood Lactate, which ISV is represented by curve 1040. As a non-limiting example the probability 1050 of this state is computed as the cumulative distribution of whole blood Lactate [p(Lactate)] being above a 2 mmol/L threshold 1051, or in some embodiments, a 4 mmol/L threshold 1051. The resulting Clinical Risk can be displayed as an index whose instantaneous time value is given by:

$$LA Index = P ( Lactate < 4 \frac{mmol}{L} \vert M ( {t}_{k} ) ) = {\int }_{- ∞}^{4} P ( Lactate \vert M ( {t}_{k} ) ) dLactate$$

The threshold 1051 in the example of FIG. 10E has different values in different embodiments. For example, other embodiments may use a threshold 1051 of 2 mmol/L, as selected or specified by a clinician.

FIG. 10F: Inadequate Oxygen Delivery

FIG. 10F illustrate the evaluation of the state of Inadequate Oxygen Delivery based on the ISV of mixed venous oxygen saturation (SvO2), which ISV is represented by curve 1040. As a non-limiting example the probability 1050 of this state is computed as the cumulative distribution of p(SvO2) less than a 40% threshold 1051. The resulting Clinical Risk can be displayed as an index whose instantaneous time value is given by:

IDO2 Index=P(SvO2<40%|M(tₖ))=∫_{-∞}⁴⁰P(SvO2|M(tₖ))dSvO2

The threshold 1051 in the example of FIG. 10F has different values in different embodiments. For example, other embodiments may use a threshold 1051 of 30%, or 50%, as selected or specified by a clinician.

FIG. 10G: Acidosis

FIG. 10G illustrates the evaluation of the state of Acidosis based on the ISV of pH of arterial blood, which ISV is represented by curve 1040. As a non-limiting example the probability of this state is computed as the cumulative distribution of arterial pH [p(pH)] less than a threshold 1051 of a pH of 7.25. The resulting Clinical Risk can be displayed as an index whose instantaneous time value is given by:

AC Index=P(pH<7.25|M(tₖ))=∫_{-∞}^{7.25}P(pH|M(tₖ))dpH

The threshold 1051 in the example of FIG. 10G has different values in different embodiments. For example, other embodiments may use a threshold 1051 of a pH of 7.00, or 7.10, or 7.3, or 7.4, or 7.5, as selected or specified by a clinician, to name but a few examples.

A listing of certain reference numbers is presented below.
100: Patient-monitoring system;
101: Patient;
102: Bedside monitors;
103: Electronic medical record;
104: Treatment device(s);
105: Laboratory Information System;
111: Computer processor;
112: Computer memory (e.g., non-transient);
113: Network interface;
121: Data reception module;
122: Physiology observer module;
123: Clinical trajectory interpreter module;
124: Display and notifications;
130: Reference material;
210: Predict model (or predict module)
211: Predicted probability density functions of internal state variables;
212: Dynamic model;
213: Estimates of probability density functions of internal state variables;
220: Update model (or update module);
221: Observation model;
230: Conditional Likelihood Kernel;
240: Initial estimates of probability density functions of internal state variables;
250: joint Probability Density Functions of the ISVs from the physiology observer module” (also known as “posterior probabilities”);
1040: Graph (curve) of an internal state variable;

Various embodiments may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:

P1. A computer-based method of risk-based monitoring of a patient, the method comprising: providing a set of sensors, each such sensor configured to be operably coupled with the patient to produce measurements of a corresponding internal state variable of the patient, the set of sensors including at least one of: (i) a heart rate sensor, and (ii) a pulse oximetry sensor; generating, by the computer, predicted probability density functions of internal state variables for a subsequent time step (tₖ₊₁), wherein the predicted probability density functions are calculated using posterior estimated probability density functions from a preceding time step (tₖ); acquiring, by a computer at subsequent time step (tₖ₊₁), physiological data from the set of sensors connected with the patient; generating a conditional likelihood kernel for the subsequent time step (tₖ₊₁), the conditional likelihood kernel comprising conditional probability density functions of the physiological data acquired at subsequent time step (tₖ₊₁) given the predicted probability density functions of internal state variables for subsequent time step (tₖ₊₁); substantially continuously estimating a risk that the patient is suffering a specific adverse medical condition, by: generating, using Bayes theorem operating on (a) the conditional likelihood kernel and (b) predicted probability density functions of internal state variables for subsequent time step (tₖ₊₁), posterior probability density functions for the plurality of the internal state variables for the subsequent time step (tₖ₊₁); and generating, for a particular internal state variable and based on the posterior probability density functions for the subsequent time step (tₖ₊₁), a probability that the particular internal state variable exceeds a corresponding pre-defined threshold for that particular internal state variable; and substantially continuously displaying, on a display device, the risk that the patient is suffering the medical specific condition.

P2. The method of P1, further comprising ascertaining that each sensor of the set of sensors is operably coupled to the patient.

P3. The method of P2, wherein ascertaining that each sensor of the set of sensors is operably coupled to the patient comprises attaching to the patient at least one sensor of the set of sensors.

P4. The method of any of P1-P3 wherein:

the specific adverse medical condition comprises inadequate oxygen delivery; and the corresponding threshold is mixed venous oxygen saturation at or below a given quantified threshold.

P5. The method of any of P1-P4 wherein: the specific adverse medical condition comprises inadequate ventilation of carbon dioxide; and the corresponding threshold is arterial partial pressure of carbon dioxide (PaCO2) at or above a given quantified arterial partial pressure of carbon dioxide threshold.

P6. The method of any of P1-P5 wherein: the specific adverse medical condition comprises acidosis; and the corresponding threshold is a blood pH below a given quantified pH threshold.

P7. The method of any of P1-P6 wherein the specific adverse medical condition comprises hyperlactatemia; and the corresponding threshold is a lactate blood level greater than given quantified lactate blood level threshold.

In any of P1-P7, the set of sensors may comprise a plurality of sensors, including without limitation the heart rate sensor, and the pulse oximetry sensor.

P8. A system for risk-based monitoring of a patient, the system comprising: a data reception module configured to receive measurements of internal state variables from sensors operably coupled to the patient; an observation model configured to produce a conditional likelihood kernel comprising conditional probability density functions of the physiological data acquired at subsequent time step (tₖ₊₁) given the predicted probability density functions of internal state variables for subsequent time step (tₖ₊₁); an inference engine configured to generate, using Bayes theorem operating on (a) the conditional likelihood kernel and (b) predicted probability density functions of internal state variables for subsequent time step (tₖ₊₁), posterior probability density functions for the plurality of the internal state variables for the subsequent time step (tₖ₊₁); a clinical trajectory interpreter module configured to generate, for a particular internal state variable and based on the posterior probability density functions for the subsequent time step (tₖ₊₁), a probability that the particular internal state variable exceeds a corresponding pre-defined threshold for that particular internal state variable; and a user interaction module configured to display, on a display device, the risk that the patient is suffering the medical specific condition.

P9. The system of P8 wherein: the specific adverse medical condition comprises inadequate oxygen delivery; and the corresponding threshold is mixed venous oxygen saturation at or below a given quantified threshold.

P10. The system of any of P8-P9 wherein: the specific adverse medical condition comprises inadequate ventilation of carbon dioxide; and the corresponding threshold is arterial partial pressure of carbon dioxide (PaCO2) at or above a given quantified threshold.

P11. The system of any of P8-P10, wherein the set of sensors further includes a respiratory rate sensor, and the measurements of internal state variables includes respiratory rate from the respiratory rate sensor.

P12. The method of any of P8-P11 wherein:

the specific adverse medical condition comprises acidosis; and

the corresponding threshold is a blood pH below a given quantified threshold.

P13. The system of any of P8-P12, wherein the set of sensors further includes a respiratory rate sensor, and the measurements of internal state variables includes respiratory rate from the respiratory rate sensor.

P14. The method of any of P8-P13 wherein: the specific adverse medical condition comprises hyperlactatemia; and the corresponding threshold is a lactate blood level greater than given quantified threshold.

In any of P8-P14, the set of sensors may comprise a plurality of sensors, including without limitation the heart rate sensor, and the pulse oximetry sensor.

P15. A non-transient computer program product comprising executable code, which executable code, when executed by a computer processor, causes the computer processor to implement a method of risk-based monitoring of a patient, the method comprising: receiving, from a set of sensors each operably coupled to the patient, measurements of a corresponding internal state variables of the patient, the set of sensors including at least one of: (i) a heart rate sensor, and (ii) a pulse oximetry sensor; generating predicted probability density functions of internal state variables for a subsequent time step (tₖ₊₁), wherein the predicted probability density functions are calculated using posterior estimated probability density functions from a preceding time step (tₖ); generating a conditional likelihood kernel for the subsequent time step (tₖ₊₁), the conditional likelihood kernel comprising conditional probability density functions of the internal state variables, based on the measurements of the corresponding internal state variables of the patient acquired at subsequent time step (tₖ₊₁) and the predicted probability density functions of internal state variables for subsequent time step (tₖ₊₁); substantially continuously estimating a risk that the patient is suffering a specific adverse medical condition, by: generating, using Bayes theorem operating on (a) the conditional likelihood kernel and (b) predicted probability density functions of internal state variables for subsequent time step (tₖ₊₁), posterior probability density functions for the plurality of the internal state variables for the subsequent time step (tₖ₊₁); and generating, for a hidden internal state variable and based on the posterior probability density functions for the subsequent time step (tₖ₊₁), a probability that the hidden internal state variable exceeds a corresponding pre-defined threshold for that particular hidden internal state variable, the probability defining a risk that the patient is suffering the adverse medical specific condition; and substantially continuously displaying, on a display device, the risk that the patient is suffering the adverse medical specific condition.

P16. The computer program product of P15, wherein: the specific adverse medical condition comprises inadequate oxygen delivery; and the corresponding threshold is mixed venous oxygen saturation at or below a given threshold.

P17. The computer program product of any of P15-P16, wherein: the specific adverse medical condition comprises inadequate ventilation of carbon dioxide; and the corresponding threshold is arterial partial pressure of carbon dioxide (PaCO2) at or above a given threshold.

P18. The system of any of P15-P17, wherein the set of sensors further includes a respiratory rate sensor, and the measurements of internal state variables includes respiratory rate from the respiratory rate sensor.

P19. The computer program product of any of P15-P18, wherein: the specific adverse medical condition comprises acidosis; and the corresponding threshold is a blood pH below a given threshold.

P20. The computer program product of any of P15-P19, wherein: the specific adverse medical condition comprises hyperlactatemia; and the corresponding threshold is a lactate blood level greater than given threshold.

In any of P15-P20, the set of sensors may comprise a plurality of sensors, including without limitation the heart rate sensor, and the pulse oximetry sensor.

In any of P1-P20, a particular internal state variable, and a hidden internal state variable, may include without limitation any of the following: inadequate oxygen delivery; inadequate ventilation of carbon dioxide; acidosis; and/or hyperlactatemia.

P101. A computer-based method of quantitatively determining the influence of a measurement of interest on a clinical patient state of a patient, the method comprising:
generating, by the computer for a time step tₖ₊₁, first joint probability density functions of a set of hidden internal state variables of the patient (“first posterior probabilities”), each joint probability density function the joint probability density functions influenced in part by a measurement of a corresponding internal state variable, from among the set of internal state variables, acquired by sensors for time step tₖ₊₁;
generating, based on the first joint probability density functions, a first plurality of clinical risks for the patient (“previously-calculated clinical risks”), each such first clinical risk comprising:
   a corresponding possible patient state for time step tₖ₊₁; and
   a corresponding probability value associated such possible patient state;
altering a set of probability density functions of the first joint probability density functions (“altered probability density functions”) to produce second joint probability density functions of the set of hidden internal state variables of the patient for time step tₖ₊₁ (“second posterior probabilities”), said altering comprising, for each probability density function of the altered probability density functions: removing the influence of a set of corresponding measurements for time step tₖ₊₁ (each a “measurement of interest”);
generating, based on the second joint probability density functions of the set of internal state variables of the patient for time step tₖ₊₁, a second plurality of clinical risks for the patient (“alternative clinical risks”), each such second clinical risk comprising:
   the corresponding possible patient state for time step tₖ₊₁; and
   a second corresponding probability value associated such possible patient state;
comparing the alternative clinical risks to the previously-calculated clinical risks to produce a quantitative measure of the importance of the set of measurements of interest;
displaying, on a display device, for each of the altered probability density functions, the importance of its corresponding set of measurements.
   P102. The computer-based method of P101, wherein:
generating, by the computer for a time step tₖ₊₁, first joint probability density functions of a set of internal state variables of the patient comprises, for each joint probability density function of the first joint probability density functions, comparing received measurements for time step tₖ₊₁ to PDFs of ISVs predicted for time step tₖ₊₁ at a previous time step tₖ; and wherein
removing, from the one of the joint probability density functions, the influence of a corresponding measurement of interest for time step tₖ₊₁ comprises substituting, for a one of the first joint probability density functions, a corresponding PDF of ISV predicted, at the previous time step tₖ, for time step tₖ₊₁.
   P103. The computer-based method of P101, wherein:
generating, by the computer for a time step tₖ₊₁, first joint probability density functions of a set of internal state variables of the patient, comprises, for each joint probability density function of the first joint probability density functions, comparing received measurements for time step tₖ₊₁ to PDFs of ISVs predicted for time step tₖ₊₁ at a previous time step tₖ; and wherein
removing, from the one of the joint probability density functions, the influence of a corresponding measurement of interest for time step tₖ₊₁ comprises substituting, for a measurement of a given internal state variable for time step tₖ₊₁, a measurement (“substitute measurement”) equal to a nominal value of the given internal variable.
   P104. The computer-based method of and of P101-P103, wherein at least one of the first joint probability density functions of a set of internal state variables of the patient corresponds to a hidden internal state variable.
   P105. The computer-based method of and of P101-P104, wherein the set of corresponding measurements for time step tₖ₊₁ comprises a single corresponding measurement.
   P106. The computer-based method of and of P101-P105, wherein the set of corresponding measurements for time step tₖ₊₁ comprises a plurality of corresponding measurements from a corresponding set of time steps preceding time step tₖ₊₁.
   P107. The computer-based method of and of P101-P106, wherein the altered probability density functions comprise a plurality of probability density functions of the first joint probability density functions.
   P108. A system for risk-based monitoring of a patient, the system comprising:
a data reception module configured to receive measurements of internal state variables from sensors operably coupled to the patient;
an observation model configured to produce a conditional likelihood kernel comprising conditional probability density functions of the physiological data acquired at subsequent time step (tₖ₊₁) given the predicted probability density functions of internal state variables for subsequent time step (tₖ₊₁);
an inference engine configured to generate, using Bayes theorem operating on (a) the conditional likelihood kernel and (b) predicted probability density functions of internal state variables for subsequent time step (tₖ₊₁), posterior probability density functions for the plurality of the internal state variables for the subsequent time step (tₖ₊₁);
a clinical trajectory interpreter module configured to generate a quantitative measure of the importance of the set of measurements of interest according to claim 1; and
a user interaction module configured to display, on a display device, the risk that the patient is suffering the medical specific condition.
   P109. A non-transient computer program product comprising executable code, which executable code, when executed by a computer processor, causes the computer processor to implement a method according to any of P101-P107.
   P201. A non-transient computer program product comprising executable code, which executable code, when executed by a computer processor, causes the computer processor to implement a method, the method comprising: substantially continuously acquiring, by the computer processor over a series of time steps t_{K}, K=0, 1, . . . Z, from a plurality of sensors connected with the patient, a set of as-measured datums m_{S}, S=1, 2 of internal state variables, including a first as-measured datum (m₁) for a first internal state variable (V₁) at time step tₖ₊₁, and a second as-measured datum (m₂) for a second internal state variable (V₂) at time step tₖ₊₁; generating, by the computer processor using the set of as-measured datums (m₁, m₂) from time step tₖ₊₁, a reference conditional likelihood kernel for the internal state variables V_{B} at time tₖ₊₁, the reference conditional likelihood kernel comprising a set of probability density functions of the internal state variables V_{B} for the time step tₖ₊₁, each of the internal state variables describing a parameter physiologically relevant to the particular patient state of said patient at time step tₖ₊₁; generating, with the computer processor and using Bayes theorem, reference posterior predicted conditional probability density functions for the plurality of the internal state variables V_{B} for the time step tₖ given the reference conditional likelihood kernel for the internal state variables V_{B} at time tₖ₊₁ and predicted probability density functions of each of the internal state variables V_{B} predicted from a preceding time step tₖ for time step tₖ₊₁; and generating, from the reference posterior predicted conditional probability density functions, a reference function of the generated internal state variable; identifying, with the computer processor, from the reference function of the generated internal state variable, a reference risk that the patient is in the particular patient state; and by editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A}); generating, by the computer processor using the first alternate set of datums, a first alternate conditional likelihood kernel for the internal state variables V_{B} at time tₖ₊₁, the first alternate conditional likelihood kernel comprising a first alternate set of probability density functions of the internal state variables V_{B} for the time step tₖ₊₁; generating, with the computer processor and using Bayes theorem, first alternate posterior predicted conditional probability density functions for the plurality of the internal state variables V_{B} for the time step tₖ₊₁ given the first alternate conditional likelihood kernel for the internal state variables V_{B} at time tₖ₊₁ and the predicted probability density functions of each of the internal state variables V_{B} for time step tₖ₊₁; generating, from the first alternate posterior predicted conditional probability density functions, a first alternate function of the generated internal state variable; and identifying, with the computer processor, from the first alternate function of the generated internal state variable, a first alternate risk that the patient is in the particular patient state at time step tₖ₊₁, said first alternate risk associated with said first internal state variable V₁; and editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A}); generating, by the computer processor using the second alternate set of datums, a second alternate conditional likelihood kernel for the internal state variables V_{B} at time tₖ₊₁, the second alternate conditional likelihood kernel comprising a second alternate set of probability density functions of the internal state variables V_{B} for the time step tₖ₊₁; generating, with the computer processor and using Bayes theorem, second alternate posterior predicted conditional probability density functions for the plurality of the internal state variables V_{B} for the time step tₖ₊₁ given the second alternate conditional likelihood kernel for the internal state variables V_{B} at time tₖ₊₁ and the predicted probability density functions of each of the internal state variables V_{B} for time step tₖ₊₁; and generating, from the second posterior predicted conditional probability density functions, a second alternate function of the generated internal state variable; and identifying, with the computer processor, from the second alternate function of the generated internal state variable, a second alternate risk that the patient is in the particular patient state at time step tₖ₊₁, said second alternate risk associated with said second internal state variable (V₂); determining, as among the as-measured datums, which as-measured datum has the quantitatively greatest influence on the reference risk (that the patient is in the particular patient state at time step tₖ₊₁) by: comparing the first alternate risk that the patient is in the particular patient state to the reference risk to produce a first delta associated with the first internal state variable, and by comparing the second alternate risk that the patient is in the particular patient state to the reference risk to produce a second delta associated with the second internal state variable, the as-measured datum having the quantitatively greatest influence on the reference risk being the as-measured datum associated with the larger of the first delta and the second delta; and displaying, on a graphical user interface, the reference risk that the patient is in the particular patient state at time step tₖ₊₁, and an identifier of the as-measured datum that has the quantitatively greatest influence on the reference risk at time step tₖ₊₁.
   P202. The system of P201, wherein: the particular patient state is hyperlactatemia; the plurality of sensors comprises a heart rate sensor and an SpO₂ sensor; the internal state variable is a hidden internal state variable: arterial lactate level; the first internal state variable (V₁) is the patient's heart rate at time step tₖ₊₁; the second internal state variable (V₂) is the patient's SpO2 at time step tₖ₊₁; and wherein: identifying, with the computer, from the function of the internal state variable, a reference risk that the patient is in the particular patient state of hyperlactatemia comprises determining the cumulative distribution of arterial lactate level above a pre-determined threshold (e.g., a pre-determined threshold of 4 mmol/L).
   P203. The system of P201, wherein: the particular patient state is inadequate ventilation of carbon dioxide; the first sensor is a heart rate sensor; the second sensor is an SpO₂ sensor; the internal state variable is a hidden internal state variable: arterial partial pressure of carbon dioxide blood [p(PaCO2)]; the first internal state variable (V₁) is the patient's heart rate at time step tₖ₊₁; the second internal state variable (V₂) is the patient's SpO2 at time step tₖ₊₁; and wherein: identifying, with the computer, from the function of the internal state variable, a reference risk that the patient is in the particular patient state of inadequate ventilation of carbon dioxide comprises determining the cumulative distribution of p(PaCO2)] above a pre-determined threshold (e.g., a pre-determined threshold of 50 mmHg).
   P204. The system of P201, wherein: the particular patient state is inadequate oxygen delivery; the first sensor is a heart rate sensor; the second sensor is an SpO₂ sensor; the third sensor is a respiratory rate sensor; the internal state variable is a hidden internal state variable: mixed venous oxygen saturation; the first internal state variable (V₁) is the patient's heart rate at time step tₖ₊₁; the second internal state variable (V₂) is the patient's SpO₂ at time step tₖ₊₁; the third internal state variable (V₃) is the patient's respiratory rate; and wherein: identifying, with the computer, from the function of the internal state variable, a reference risk that the patient is in the particular patient state of inadequate oxygen delivery comprises determining the cumulative distribution of cumulative distribution mixed venous oxygen saturation below a pre-determined threshold (e.g., a pre-determined threshold of 40%).
   P205. The system of P201, wherein: the particular patient state is acidosis; the first sensor is a heart rate sensor; he second sensor is an SpO₂ sensor; the third sensor is a respiratory rate sensor; the internal state variable is a hidden internal state variable: arterial blood pH; the first internal state variable (V₁) is the patient's heart rate at time step tₖ₊₁; the second internal state variable (V₂) is the patient's SpO2 at time step tₖ₊₁; the third internal state variable (V₃) is the patient's respiratory rate; and wherein: identifying, with the computer, from the function of the internal state variable, a reference risk that the patient is in the particular patient state of acidosis comprises determining the cumulative distribution of Arterial blood pH below a pre-determined threshold (e.g., a pre-determined threshold of 7.25).

Various embodiments of this disclosure may be implemented at least in part in any conventional computer programming language. For example, some embodiments may be implemented in a procedural programming language (e.g., “C”), or in an object-oriented programming language (e.g., “C++”), or in Python, R, Java, LISP or Prolog. Other embodiments of this disclosure may be implemented as preprogrammed hardware elements (e.g., application specific integrated circuits, FPGAs, and digital signal processors), or other related components.

In an alternative embodiment, the disclosed apparatus and methods may be implemented as a computer program product for use with a computer system. Such implementation may include a series of computer instructions fixed either on a tangible medium, such as a non-transient computer readable medium (e.g., a diskette, CD-ROM, ROM, FLASH memory, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of this disclosure may be implemented as a combination of both software (e.g., a computer program product) and hardware. Still other embodiments of this disclosure are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads.

The embodiments described above are intended to be merely exemplary; numerous variations and modifications will be apparent to those skilled in the art. All such variations and modifications are intended to be within the scope of the present disclosure as defined in any appended claims.

Various examples and embodiments consistent with the present disclosure have be described in detailed above. It is to be understood that these examples and embodiments of the present disclosure are provided for exemplary and illustrative purposes only. Various modifications and changes may be made to the disclosed embodiments by persons skilled in the art without departing from the scope of the present disclosure as defined in the appended claims.

What is claimed is:

1. A method of transforming measured data of a patient into data for a particular patient state based on a generated internal state variable, the method comprising:
providing a plurality of sensors including at least a first sensor and a second sensor, to measure a corresponding plurality of internal state variables, the plurality of sensors physically attached to the patient;
substantially continuously acquiring, by a computer over a series of time steps t_{K}, K=0, 1, . . . Z, from the plurality of sensors connected with the patient, a set of as-measured datums m_{S}, S=1, 2 of internal state variables, including a first as-measured datum (m₁) for a first internal state variable (V₁) at time step tₖ₊₁, and a second as-measured datum (m₂) for a second internal state variable (V₂) at time step tₖ₊₁;
generating, by the computer using the set of as-measured datums from time step tₖ₊₁, a reference conditional likelihood kernel for the internal state variables at time tₖ₊₁, the reference conditional likelihood kernel comprising a set of probability density functions of the internal state variables for the time step tₖ₊₁, each of the internal state variables describing a parameter physiologically relevant to the particular patient state of said patient at time step tₖ₊₁;
generating, with the computer and using Bayes theorem, reference posterior predicted conditional probability density functions for the plurality of the internal state variables for the time step tₖ given the reference conditional likelihood kernel for the internal state variables at time tₖ₊₁ and predicted probability density functions of each of the internal state variables predicted from a preceding time step tₖ for time step tₖ₊₁; and
generating, from the reference posterior predicted conditional probability density functions, a reference function of the generated internal state variable;
identifying, with the computer, from the reference function of the generated internal state variable, a reference risk that the patient is in the particular patient state;
and by
editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A});
generating, by the computer using the first alternate set of datums, a first alternate conditional likelihood kernel for the internal state variables at time tₖ₊₁, the first alternate conditional likelihood kernel comprising a first alternate set of probability density functions of the internal state variables for the time step tₖ₊₁;
generating, with the computer and using Bayes theorem, first alternate posterior predicted conditional probability density functions for the plurality of the internal state variables for the time step tₖ₊₁ given the first alternate conditional likelihood kernel for the internal state variables at time tₖ₊₁ and the predicted probability density functions of each of the internal state variables for time step tₖ₊₁;
generating, from the first alternate posterior predicted conditional probability density functions, a first alternate function of the generated internal state variable; and
identifying, with the computer, from the first alternate function of the generated internal state variable, a first alternate risk that the patient is in the particular patient state at time step tₖ₊₁, said first alternate risk associated with said first internal state variable V₁; and
editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A});
generating, by the computer using the second alternate set of datums, a second alternate conditional likelihood kernel for the internal state variables at time tₖ₊₁, the second alternate conditional likelihood kernel comprising a second alternate set of probability density functions of the internal state variables for the time step tₖ₊₁;
generating, with the computer and using Bayes theorem, second alternate posterior predicted conditional probability density functions for the plurality of the internal state variables for the time step tₖ₊₁ given the second alternate conditional likelihood kernel for the internal state variables at time tₖ₊₁ and the predicted probability density functions of each of the internal state variables for time step tₖ₊₁; and
generating, from the second posterior predicted conditional probability density functions, a second alternate function of the generated internal state variable; and
identifying, with the computer, from the second alternate function of the generated internal state variable, a second alternate risk that the patient is in the particular patient state at time step tₖ₊₁, said second alternate risk associated with said second internal state variable (V₂);
determining, as among the as-measured datums, which as-measured datum has the quantitatively greatest influence on the reference risk (that the patient is in the particular patient state at time step tₖ₊₁) by:
comparing the first alternate risk that the patient is in the particular patient state to the reference risk to produce a first delta associated with the first internal state variable, and by
comparing the second alternate risk that the patient is in the particular patient state to the reference risk to produce a second delta associated with the second internal state variable,
the as-measured datum having the quantitatively greatest influence on the reference risk being the as-measured datum associated with the larger of the first delta and the second delta; and
displaying, on a graphical user interface, the reference risk that the patient is in the particular patient state at time step tₖ₊₁, and an identifier of the as-measured datum has the quantitatively greatest influence on the reference risk at time step tₖ₊₁.

2. The method of claim 1, wherein:
the particular patient state is hyperlactatemia;
the first sensor is a heart rate sensor;
the second sensor is an SpO₂ sensor;
the internal state variable is a hidden internal state variable: whole blood lactate level;
the first internal state variable (V₁) is the patient's heart rate at time step tₖ₊₁;
the second internal state variable (V₂) is the patient's SpO2 at time step tₖ₊₁; and wherein:
identifying, with the computer, from the function of the internal state variable, a reference risk that the patient is in the particular patient state of hyperlactatemia comprises determining the cumulative distribution of whole blood lactate level above a predetermined threshold.

3. The method of claim 2, wherein the first alternate datum value to produce a first alternate datum (m_{1A}) comprises a datum value selected from one of a nominal heart and a null value for the heart rate.

4. The method of claim 2, wherein the second alternate datum value to produce a second alternate datum (m_{2A}) comprises a datum value selected from one of: a nominal value of SpO2 and a null value of SpO2.

5. The method of claim 1, wherein:
the particular patient state is inadequate ventilation of carbon dioxide;
the first sensor is a heart rate sensor;
the second sensor is an SpO₂ sensor;
the internal state variable is a hidden internal state variable: arterial partial pressure of carbon dioxide blood [p(PaCO2)];
the first internal state variable (V₁) is the patient's heart rate at time step tₖ₊₁;
the second internal state variable (V₂) is the patient's SpO₂ at time step tₖ₊₁; and wherein:
identifying, with the computer, from the function of the internal state variable, a reference risk that the patient is in the particular patient state of inadequate ventilation of carbon dioxide comprises determining the cumulative distribution of p(PaCO2)] above a predetermined threshold.

6. The method of claim 5, wherein the first alternate datum value to produce a first alternate datum (m1A) comprises a datum value selected from one of a nominal heart and a null value for the heart rate.

7. The method of claim 5, wherein the second alternate datum value to produce a second alternate datum (m2A) comprises a datum value selected from one of: a nominal value of SpO2 and a null value of SpO2.

8. The method of claim 1, wherein the set of sensors further comprises a third sensor, and wherein:
the particular patient state is acidosis;
the first sensor is a heart rate sensor;
the second sensor is an SpO₂ sensor;
the third sensor is a respiratory rate sensor;
the internal state variable is a hidden internal state variable: arterial blood pH;
the first internal state variable (V₁) is the patient's heart rate at time step tₖ₊₁;
the second internal state variable (V₂) is the patient's SpO2 at time step tₖ₊₁;
the third internal state variable (V₃) is the patient's respiratory rate; and wherein:
identifying, with the computer, from the function of the internal state variable, a reference risk that the patient is in the particular patient state of acidosis comprises determining the cumulative distribution of Arterial blood pH below a predetermined threshold.

9. The method of claim 8, wherein the first alternate datum value to produce a first alternate datum (m1A) comprises a datum value selected from one of a nominal heart and a null value for the heart rate.

10. The method of claim 8, wherein the second alternate datum value to produce a second alternate datum (m2A) comprises a datum value selected from one of: a nominal value of SpO2 and a null value of SpO₂.

11. The method of claim 8, wherein:
substantially continuously acquiring, by a computer over a series of time steps t_{K}, K=0, 1, . . . Z, from the plurality of sensors connected with the patient, a set of as-measured datums m_{S}, S=1, 2 of internal state variables further includes acquiring a third as-measured datum (m₃) for a third internal state variable (V₃) at time step tₖ₊₁; and the method further comprises:
editing the set of as-measured datums by replacing the third as-measured datum (m₃) with a third alternate datum value to produce a third alternate datum (m_{3A}), the third alternate datum value distinct from the as-measured value of the third as-measured datum (m₃), to produce a third alternate set of datums including the first as-measured datum (m₁) and the second as-measured datum (m₂) and the third alternate datum (m_{3A});
generating, by the computer using the third alternate set of datums, a third alternate conditional likelihood kernel for the internal state variables at time tₖ₊₁, the third alternate conditional likelihood kernel comprising a third alternate set of probability density functions of the internal state variables for the time step tₖ₊₁;
generating, with the computer and using Bayes theorem, third alternate posterior predicted conditional probability density functions for the plurality of the internal state variables for the time step tₖ₊₁ given the third alternate conditional likelihood kernel for the internal state variables at time tₖ₊₁ and the predicted probability density functions of each of the internal state variables for time step tₖ₊₁;
generating, from the third alternate posterior predicted conditional probability density functions, a third alternate function of the generated internal state variable; and
identifying, with the computer, from the third alternate function of the generated internal state variable, a third alternate risk that the patient is in the particular patient state at time step tₖ₊₁, said third alternate risk associated with said third internal state variable V₃; and wherein:
determining, as among the as-measured datums, which as-measured datum has the quantitatively greatest influence on the reference risk (that the patient is in the particular patient state at time step tₖ₊₁) comprises:
comparing the first alternate risk that the patient is in the particular patient state to the reference risk to produce a first delta associated with the first internal state variable, and by
comparing the second alternate risk that the patient is in the particular patient state to the reference risk to produce a second delta associated with the second internal state variable,
comparing the third alternate risk that the patient is in the particular patient state to the reference risk to produce a third delta associated with the first internal state variable, and wherein
the as-measured datum having the quantitatively greatest influence on the reference risk being the as-measured datum associated with the larger of the first delta and the second delta and the third delta.

12. The method of claim 1, wherein the set of sensors further comprises a third sensor, and wherein:
the particular patient state is inadequate oxygen delivery;
the first sensor is a heart rate sensor;
the second sensor is an SpO₂ sensor;
the third sensor is a respiratory rate sensor;
the internal state variable is a hidden internal state variable: mixed venous oxygen saturation;
the first internal state variable (V₁) is the patient's heart rate at time step tₖ₊₁;
the second internal state variable (V₂) is the patient's SpO2 at time step tₖ₊₁;
the third internal state variable (V₃) is the patient's respiratory rate; and wherein:
identifying, with the computer, from the function of the internal state variable, a reference risk that the patient is in the particular patient state of inadequate oxygen delivery comprises determining the cumulative distribution of cumulative distribution mixed venous oxygen saturation below a predetermined threshold.

13. The method of claim 12, wherein the first alternate datum value to produce a first alternate datum (m1A) comprises a datum value selected from one of a nominal heart and a null value for the heart rate.

14. The method of claim 12, wherein the second alternate datum value to produce a second alternate datum (m2A) comprises a datum value selected from one of: a nominal value of SpO2 and a null value of SpO2.

15. The method of claim 12, wherein:
substantially continuously acquiring, by a computer over a series of time steps t_{K}, K=0, 1, . . . Z, from the plurality of sensors connected with the patient, a set of as-measured datums m_{S}, S=1, 2 of internal state variables further includes acquiring a third as-measured datum (m₃) for a third internal state variable (V₃) at time step tₖ₊₁; and the method further comprises:
editing the set of as-measured datums by replacing the third as-measured datum (m₃) with a third alternate datum value to produce a third alternate datum (m_{3A}), the third alternate datum value distinct from the as-measured value of the third as-measured datum (m₃), to produce a third alternate set of datums including the first as-measured datum (m₁) and the second as-measured datum (m₂) and the third alternate datum (m_{3A});
generating, by the computer using the third alternate set of datums, a third alternate conditional likelihood kernel for the internal state variables at time tₖ₊₁, the third alternate conditional likelihood kernel comprising a third alternate set of probability density functions of the internal state variables for the time step tₖ₊₁;
generating, with the computer and using Bayes theorem, third alternate posterior predicted conditional probability density functions for the plurality of the internal state variables for the time step tₖ₊₁ given the third alternate conditional likelihood kernel for the internal state variables at time tₖ₊₁ and the predicted probability density functions of each of the internal state variables for time step tₖ₊₁;
generating, from the third alternate posterior predicted conditional probability density functions, a third alternate function of the generated internal state variable; and
identifying, with the computer, from the third alternate function of the generated internal state variable, a third alternate risk that the patient is in the particular patient state at time step tₖ₊₁, said third alternate risk associated with said third internal state variable V₃; and wherein:
determining, as among the as-measured datums, which as-measured datum has the quantitatively greatest influence on the reference risk (that the patient is in the particular patient state at time step tₖ₊₁) comprises:
comparing the first alternate risk that the patient is in the particular patient state to the reference risk to produce a first delta associated with the first internal state variable, and by
comparing the second alternate risk that the patient is in the particular patient state to the reference risk to produce a second delta associated with the second internal state variable,
comparing the third alternate risk that the patient is in the particular patient state to the reference risk to produce a third delta associated with the first internal state variable, and wherein
the as-measured datum having the quantitatively greatest influence on the reference risk being the as-measured datum associated with the larger of the first delta and the second delta and the third delta.

16. A system for transforming measured data of a patient into data for a particular patient state based on a generated internal state variable, the system comprising:
a computer comprising a computer processor;
a display in data communication with the computer processor;
a memory in data communication with the computer processor, the memory holding instructions that, when executed by the computer processor, cause the system to perform a method, the method comprising:
substantially continuously acquiring, by a computer over a series of time steps t_{K}, K=0, 1, . . . Z, from a plurality of sensors connected with the patient, a set of as-measured datums m_{S}, S=1, 2 of internal state variables, including a first as-measured datum (m₁) for a first internal state variable (V₁) at time step tₖ₊₁, and a second as-measured datum (m₂) for a second internal state variable (V₂) at time step tₖ₊₁;
generating, by the computer using the set of as-measured datums (m₁, m₂) from time step tₖ₊₁, a reference conditional likelihood kernel for the internal state variables at time tₖ₊₁, the reference conditional likelihood kernel comprising a set of probability density functions of the internal state variables for the time step tₖ₊₁, each of the internal state variables describing a parameter physiologically relevant to the particular patient state of said patient at time step tₖ₊₁;
generating, with the computer and using Bayes theorem, reference posterior predicted conditional probability density functions for the plurality of the internal state variables for the time step tₖ given the reference conditional likelihood kernel for the internal state variables at time tₖ₊₁ and predicted probability density functions of each of the internal state variables predicted from a preceding time step tₖ for time step tₖ₊₁; and
generating, from the reference posterior predicted conditional probability density functions, a reference function of the generated internal state variable;
identifying, with the computer, from the reference function of the generated internal state variable, a reference risk that the patient is in the particular patient state;
and by
editing the set of as-measured datums by replacing the first as-measured datum (m₁) with a first alternate datum value to produce a first alternate datum (m_{1A}), the first alternate datum value distinct from the as-measured value of the first as-measured datum (m₁), to produce a first alternate set of datums including the second as-measured datum (m₂) and the first alternate datum (m_{1A});
generating, by the computer using the first alternate set of datums, a first alternate conditional likelihood kernel for the internal state variables at time tₖ₊₁, the first alternate conditional likelihood kernel comprising a first alternate set of probability density functions of the internal state variables for the time step tₖ₊₁;
generating, with the computer and using Bayes theorem, first alternate posterior predicted conditional probability density functions for the plurality of the internal state variables for the time step tₖ₊₁ given the first alternate conditional likelihood kernel for the internal state variables at time tₖ₊₁ and the predicted probability density functions of each of the internal state variables for time step tₖ₊₁;
generating, from the first alternate posterior predicted conditional probability density functions, a first alternate function of the generated internal state variable; and
identifying, with the computer, from the first alternate function of the generated internal state variable, a first alternate risk that the patient is in the particular patient state at time step tₖ₊₁, said first alternate risk associated with said first internal state variable V₁; and
editing the set of as-measured datums by replacing the second as-measured datum (m₂) with a second alternate datum value to produce a second alternate datum (m_{2A}), the second alternate datum value distinct from the as-measured value for the second as-measured datum (m₂), to produce a second alternate set of datums including the first as-measured datum (m₁) and the second alternate datum (m_{2A});
generating, by the computer using the second alternate set of datums, a second alternate conditional likelihood kernel for the internal state variables at time tₖ₊₁, the second alternate conditional likelihood kernel comprising a second alternate set of probability density functions of the internal state variables for the time step tₖ₊₁;
generating, with the computer and using Bayes theorem, second alternate posterior predicted conditional probability density functions for the plurality of the internal state variables for the time step tₖ₊₁ given the second alternate conditional likelihood kernel for the internal state variables at time tₖ₊₁ and the predicted probability density functions of each of the internal state variables for time step tₖ₊₁; and
generating, from the second posterior predicted conditional probability density functions, a second alternate function of the generated internal state variable; and
identifying, with the computer, from the second alternate function of the generated internal state variable, a second alternate risk that the patient is in the particular patient state at time step tₖ₊₁, said second alternate risk associated with said second internal state variable (V₂);
determining, as among the as-measured datums, which as-measured datum has the quantitatively greatest influence on the reference risk (that the patient is in the particular patient state at time step tₖ₊₁) by:
comparing the first alternate risk that the patient is in the particular patient state to the reference risk to produce a first delta associated with the first internal state variable, and by
comparing the second alternate risk that the patient is in the particular patient state to the reference risk to produce a second delta associated with the second internal state variable,
the as-measured datum having the quantitatively greatest influence on the reference risk being the as-measured datum associated with the larger of the first delta and the second delta; and
displaying, on a graphical user interface, the reference risk that the patient is in the particular patient state at time step tₖ₊₁, and an identifier of the as-measured datum that has the quantitatively greatest influence on the reference risk at time step tₖ+1.

17. The system of claim 16, wherein:
the particular patient state is hyperlactatemia;
the plurality of sensors comprises a heart rate sensor and an SpO₂ sensor;
the internal state variable is a hidden internal state variable: whole blood lactate level;
the first internal state variable (V₁) is the patient's heart rate at time step tₖ₊₁;
the second internal state variable (V₂) is the patient's SpO2 at time step tₖ₊₁; and wherein:
identifying, with the computer, from the function of the internal state variable, a reference risk that the patient is in the particular patient state of hyperlactatemia comprises determining the cumulative distribution of whole blood lactate level above a threshold of 4 mmol/L.

18. The system of claim 16, wherein:
the particular patient state is inadequate ventilation of carbon dioxide;
the first sensor is a heart rate sensor;
the second sensor is an SpO₂ sensor;
the internal state variable is a hidden internal state variable: arterial partial pressure of carbon dioxide blood [p(PaCO2)];
the first internal state variable (V₁) is the patient's heart rate at time step tₖ₊₁;
the second internal state variable (V₂) is the patient's SpO2 at time step tₖ₊₁; and wherein:
identifying, with the computer, from the function of the internal state variable, a reference risk that the patient is in the particular patient state of inadequate ventilation of carbon dioxide comprises determining the cumulative distribution of p(PaCO2)] above a threshold of 50 mmHg.

19. The system of claim 16, wherein:
the particular patient state is inadequate oxygen delivery;
the first sensor is a heart rate sensor;
the second sensor is an SpO₂ sensor;
the third sensor is a respiratory rate sensor;
the internal state variable is a hidden internal state variable: mixed venous oxygen saturation;
the first internal state variable (V₁) is the patient's heart rate at time step tₖ₊₁;
the second internal state variable (V₂) is the patient's SpO2 at time step tₖ₊₁;
the third internal state variable (V₃) is the patient's respiratory rate; and wherein:
identifying, with the computer, from the function of the internal state variable, a reference risk that the patient is in the particular patient state of inadequate oxygen delivery comprises determining the cumulative distribution of cumulative distribution mixed venous oxygen saturation below 40%.

20. The system of claim 16, wherein:
the particular patient state is acidosis;
the first sensor is a heart rate sensor;
the second sensor is an SpO₂ sensor;
the third sensor is a respiratory rate sensor;
the internal state variable is a hidden internal state variable: arterial blood pH;
the first internal state variable (V₁) is the patient's heart rate at time step tₖ₊₁;
the second internal state variable (V₂) is the patient's SpO2 at time step tₖ₊₁;
the third internal state variable (V₃) is the patient's respiratory rate; and wherein:
identifying, with the computer, from the function of the internal state variable, a reference risk that the patient is in the particular patient state of acidosis comprises determining the cumulative distribution of arterial blood pH below a threshold of 7.25.
